(19) United States
(12) Patent Application Publication
(10) Pub. No.: US 2017/0050043 A1
(43) Pub. Date: Feb. 23, 2017

(54) PROBE COMPRISING OPTICALLY DIFFUSING FIBER, METHOD FOR MANUFACTURING SAME AND APPLICATIONS THEREOF

(71) Applicant: PUKYONG NATIONAL UNIVERSITY INDUSTRY-UNIVERSITY COOPERATION FOUNDATION, Nam-gu, Busan (KR)

(72) Inventors: HYUN WOOK KANG, Nam-gu, Busan (KR); YEH CHAN AHN, Nam-gu, Busan (KR); KANG DAE LEE, Saha-gu, Busan (KR); HYOUNG SHIN LEE, Nam-gu, Busan (KR); MIN WOO AHN, Saha-gu, Busan (KR)

(73) Assignee: YUKYONG NATIONAL UNIVERSITY INDUSTRY-UNIVERSITY COOPERATION FOUNDATION, Nam-gu, Busan (KR)

(21) Appl. No.: 14/915,847

(22) PCT Filed: Dec. 8, 2014
(86) PCT No.: PCT/KR2014/012022

§ 371 Date: Mar. 1, 2016

(30) Foreign Application Priority Data
Apr. 18, 2014 (KR) 10-2014-0046881
Aug. 29, 2014 (KR) 10-2014-0114243
Sep. 15, 2014 (KR) 10-2014-0121830
Nov. 13, 2014 (KR) 10-2014-0157934

(51) Int. Cl. A61N 5/06; A61M 29/02; A61B 5/00; A61M 25/10

(52) U.S. Cl. CPC A61N 5/0601; A61M 25/1002; A61M 25/1018; A61M 29/02; A61B 5/0066; A61B 5/0084; A61N 5/062; A61N 5/0625; A61B 5/4836; A61B 18/24

(57) ABSTRACT

The present invention relates to an optically diffusing fiber probe, a method for manufacturing the same, and an application thereof. More specifically, the present invention relates to an optically diffusing fiber probe capable of emitting light in a plurality of directions and a method for manufacturing the same, hybrid optical medical equipment for both diagnosis and treatment of tubular human tissue, a catheter-based laser treatment device, and an electromagnetic energy application device for tubular tissue stricture, comprising the optically diffusing fiber probe.

### CROSS-REFERENCE TO RELATED APPLICATIONS

This application is the national stage for International Patent Cooperation Treaty Application PCT/KR2014/012022, filed Dec. 8, 2014, which claims priority from Korean Patent Application No. 10-2014-0046881, filed on Apr. 18, 2014, in the Korean Intellectual Property Office; Korean Patent Application No. 10-2014-0114243, filed on Aug. 29, 2014, in the Korean Intellectual Property Office; Korean Patent Application No. 10-2014-0121830, filed on Sep. 15, 2014, in the Korean Intellectual Property Office; and Korean Patent Application No. 10-2014-0157934, filed on Nov. 13, 2014, also in the Korean Intellectual Property Office. The entire contents of said applications are incorporated herein by reference for all purposes.

### BACKGROUND OF THE INVENTION

(a) Field of the Invention

The present invention relates to an optically diffusing fiber, a probe comprising the optically diffusing fiber, a method for manufacturing the same, and an optical fiber application device thereof. More specifically, the present invention relates to an optically diffusing fiber and an optically diffusing fiber probe capable of emitting light in a plurality of directions, a method for manufacturing the same, hybrid optical medical equipment for both diagnosis and treatment of tubular human tissue, a catheter-based laser treatment device, and an electromagnetic energy application device for tubular tissue stricture, comprising the optically diffusing fiber probe.

(b) Description of the Related Art

In general, optical fiber probe devices which emit light delivered through an inner core are widely utilized in various medical fields, and side surface emission type or front emission type optical fiber probes are mainly used. However, light emission in a fixed direction causes spatial restriction when treating an inner tissue of a human body.

Upon reviewing Korean and foreign markets for laser treatment, as the market in Korea focuses on the treatment for dermatological diseases, the use or development of optical fibers is in a poor condition. However, recently, as the demand for minimally invasive surgery increases and the market grows, an interest for the development of optical fibers increases. In the case of foreign markets, many investments in the development of front or side type optical fiber are done. The optical fibers are used for clinical treatments, for example, treatment for prostate, liposuction, treatment for periodontal diseases, etc.

However, most optical fiber probes emit light only in one direction. Thus, it is necessary to develop optical fiber probes for delivering electromagnet energy in various directions or in a fixed direction.

Asthma, which is a sort of tracheobronchial diseases, is an allergic disease caused by an allergic inflammatory response of sensitive bronchus. Asthma refers to a disease showing the following symptoms: Bronchial mucous membrane swells up by inflammation of bronchus forming an airway, bronchial muscle falls into a fit of convulsion, bronchus becomes narrow or blocked, which leads to dyspnea, wheezing and severe coughing. Due to environmental factors, more than 3 hundred million people in the world suffer from acute exacerbation of asthma, and every year, more than 250 thousand people die of the disease (2007, WHO). In the case of USA, for example, one person in the US spends about 3.7 million won for the treatment of asthma, and a total of 60 trillion won or more are estimated (2011, CDC). In the case of Korea, according to the Health Insurance Analysis Statistics in 2010 issued by the National Health Insurance Service, the number of patients with asthma gradually increases by 15% or more annually. Currently, it is estimated that more than 2.35 million people suffer from asthma, and annual socioeconomic costs incurred for asthma exceeds about 2.5 trillion won (2005, Korea Asthma Allergy Foundation).

In order to alleviate or treat symptoms of asthma, suction-type asthma therapeutic agents such as singulair or seretide, or oral-type asthma therapeutic agents are generally used. However, this kind of treatment using medicines temporarily alleviates symptoms. Since asthma needs to be continuously treated for a long time, the costs for treating asthma increases, it is inconvenient for patients with asthma, and side-effects and allergic reactions are often induced.

As a means for improving the above problems, RF surgery equipment for treating asthma was developed. In this regard, the Boston Scientific Corporation invented EP 01803409 entitled “System for treating tissue with radio frequency vascular electrode array.” Bronchial thermoplasty (product name) by the Boston Scientific Corporation involves induction of thermotherapy by delivering RF energy to the tissue where asthma occurs, by using a catheter, that is, Bronchial thermoplasty relates to a method delivering energy to a body tissue based on the delivery of electric current. However, Bronchial thermoplasty is expensive because of monopolistic supply of equipment, and accordingly, the costs for asurgery exceed 20 million won. Additionally, due to non-uniformimpedance within the body tissue, thermal damage often occurs. Accordingly, recovery is slow, pain is great, and recurrence rate is high, which gives a great burden on patients and medical systems.

Conventional methods for treating trachea include tracheal resection, balloon dilation, stenting and surgery using tracheostomy tube (T-tube), etc. As to the conventional methods for treating trachea, due to the occurrence of scar by an invasive surgery, there is a high possibility that tracheal stricture could recur. Additionally, the conventional methods cause damage on surrounding tissues due to hemorrhage or photothermal treatment, and have a high risk in inflammation and infection. Thus, most of them simply exhibit temporary treatment effects. In the case of the balloon dilation, a fixed size of airway may be temporarily secured by the expansion of balloon. However, due to contraction of tissue, re-stricture could easily occur. Also, a result of surgery and a period for recovery greatly depend on skills and experience of an operator.

Thus, it is urgently necessary to develop treatment equipment capable of performing medicinal treatment in combination therewith, in order todecreasea recurrence rate of contraction and minimize complications such as inflammation, infection, etc. which could occur during recovery, by expanding the part of trachea where stricture occurs and permanently modifying a structure of tissue simultaneously.

Meanwhile, a conventional laser treatment uses a method of inserting an optical fiber delivering laser into a varix and generating heat using optical energy, thereby contracting blood vessels and detouring obstructed blood flow. However, in order to minimize thermal damage and medical accidents, a user who uses laser treatment equipment needs to have a lot of surgery experience and high surgery capacity, and thus this treatment is restrictive and difficult to be performed. Especially, the conventional laser treatment causes intravascular perforation by optical fiber which directly contacts a blood vessel or lacks uniform thermal delivery, and thus recurrence and medical accidents occur due to insufficient treatment or excessive treatment.

In general, before performing trachea surgery, information on the degree of stricture in the trachea is obtained using a computed tomography (CT). However, CT has a limitation that a prognosis cannot be accurately and rapidly monitored therefrom. Thus, it is necessary to provide a diagnosis means capable of increasing treatment efficiency and securing stability, by obtaining a change in tissue right after treatment through real-time imaging according to depth and length of stricture of trachea.

### SUMMARY OF THE INVENTION

The present invention is to solve the conventional problems as above. The present invention aims to provide a probe comprising an optically diffusing fiber capable of emitting light in a plurality of directions unlike the conventional optical fiber, and thus capable of constantly emitting electromagnetic energy in a plurality of directions to tubular tissue diseases or solid cancers, such as thyroid cancer, breast cancer, kidney cancer, etc., to treat a broader range of diseases in a safe and efficient way, and a method for manufacturing the same.

Also, the present invention aims to provide hybrid optical medical equipment for both diagnosis and treatment of a tubular human tissue, capable of inducing photothermal treatment for tubular human tissues, such as trachea, blood vessel, ureter, etc., by a single human activating module including an optically diffusing fiber installed to penetrate into the inside of the probe, and performing real-time monitoring on an OCT image for the human tissue during a process for inducing photothermal treatment, thereby allowing integrated diagnosis and induction of treatment for a lesion tissue to be made while minimizing the damage on the human tissue.

Also, the present invention aims to provide hybrid optical medical equipment for both diagnosis and treatment of a tubular human tissue capable of performing macroscopic monitoring of a tubular human tissue using a camera and an optical source module for photographing during the process of inducing laser photothermal treatment using an optically diffusing fiber, a side type optical fiber arranged in a predetermined pattern, a single mode optical fiber, etc. and microscopic monitoring of a tubular human tissue by obtaining an OCT image, thereby allowing precise diagnosis and induction of treatment for an initial lesion in the tubular tissue.

Also, the present invention aims to provide a catheter-based laser treatment device capable of preventing recurrence of trachea stricture, minimizing complications such as inflammation, infection, etc. which may occur during recovery, and treating a part to be treated during treatment while monitoring the part in real-time.

Also, the present invention aims to provide an electromagnetic energy application device for tubular tissue stricture capable of minimizing hemorrhage through blood vessels before and after and during the treatment by using an expansion of various balloon catheters with a geometric shape, inducing blood vessel stricture without contraction of balloon catheter, and including a fixed shape of balloon catheter from which deflation may be induced according to vasoconstriction during laser treatment.

The above-described objects may be achieved by technical resolutions below.

An optically diffusing fiber, including: a fabrication length of a tissue treatment section required for laser treatment; a tapering angle and an end diameter within the fabrication length capable of uniformly delivering optical energy; a fabrication angle and fabrication part interval capable of varying optical energy distribution delivered; and a height of an optically diffusing surface fabricated to vary a diffusion range of optical energy.

An optical fiber probe for treating a tubular tissue disease or a solid cancer including the optically diffusing fiber.

A method for manufacturing an optically diffusing fiber probe, which includes the steps of (a) inputting fabrication values including an optically diffusing range according to a disease part to be treated, energy distribution, optical fiber fabrication length, tapering angle, end diameter, fabrication angle, fabrication part interval, and height of an optically diffusing surface for manufacturing a suitable optical fiber for treatment length, etc.; (b) outputting a fabrication control signal through a fabrication controlling part; (c) fabricating a side surface and front end of an optical fiber by moving the optical fiber in the rotational direction and front and back direction according to the fabrication control signal; (d) delivering optical energy to an optical fiber; (e) measuring optical energy delivered to the side surface and front end of an optical fiber through a side surface optical sensor and a front optical sensor; and (f) determining whether to go through additional fabrication and polishing by comparing the measured strength and the pre-stored energy distribution of the optical fiber.

The method for manufacturing an optically diffusing fiber probe, wherein the step (f) further includes the step of conducting a feedback for precise fabrication when determined to go through an additional fabrication, and fabrication delivery speed, rotational speed, and fabrication energy are minutely controlled during the precise fabrication.

The method for manufacturing an optically diffusing fiber probe, wherein the step (a) of inputting the fabrication values further includes the step (a-1) of controlling the fabrication length L of the optical fiber in consideration of the tissue treatment section required for laser treatment, and the step (a-1) determines an initial fabrication location of the optical fiber with an overall fabrication length in consideration of a translational stage.

The method for manufacturing an optically diffusing fiber probe, wherein the step (a) of inputting the fabrication values for the optical fiber to be fabricated further includes the step (a-2) of determining the tapering angle α and end diameter d of the optical fiber so that light of the optical energy is uniformly delivered through the optical fiber, and the step (a-2) determines the tapering angle α and end diameter d of the optical fiber by simultaneously or independently controlling the translational speed, rotational speed, power of fabrication energy source (0.1 W to 50 W), and area of energy source of the optical fiber.

The method for manufacturing an optically diffusing fiber probe, wherein the step (a) of inputting fabrication values for the optical fiber to be fabricated further includes the step (a-3) of determining a fabrication angle β and a fabrication part interval w to vary the optical energy distribution delivered through the optical fiber, and the step (a-3) determines the fabrication angle β and fabrication part interval w by simultaneously or independently controlling the translational speed and rotational speed of the optical fiber.

The method for manufacturing an optically diffusing fiber probe of the above 7, wherein the step (a) of inputting fabrication values for the optical fiber to be fabricated further includes the step (a-4) of determining the height p of the optically diffusing surface to vary the diffusion range of optical energy light through the optical fiber, and the step (a-4) determines the height p of the optically diffusing surface by controlling the rotational speed of the optical fiber, power of fabrication energy source (0.1 W to 50 W) and area of energy source.

Hybrid optical medical equipment for both diagnosis and treatment of a tubular human tissue, which includes a probe moving by being inserted in a tubular human tissue; a human activating optical fiber module protruding to the front end of the probe by passing an inner passage of the probe, the human activating optical fiber module performing any one selected from obtaining an optical coherence tomography (OCT) image of a tubular human tissue through infrared light emission of a predetermined wavelength area and inducing tubular human tissue photothermal treatment through laser emission; a controller connected to the human activating optical fiber module, performing the operation control of the human activating optical fiber module for obtaining an OCT image of human tissue and for inducing human tissue photothermal treatment; and an OCT image output device connected to the controller, outputting an OCT image obtained from the human activating optical fiber module, wherein OCT image monitoring on the tubular human tissue and laser stimulation thereon are performed integrally.

The hybrid optical medical equipment for both diagnosis and treatment of a tubular human tissue, wherein the human activating optical fiber module performing tubular human tissue photothermal treatment inducement through the laser emission includes an optically diffusing fiber.

The hybrid optical medical equipment for both diagnosis and treatment of a tubular human tissue, wherein the human activating optical fiber module includes an optical fiber for diagnosis emitting near infrared ray in a wavelength range of 800 to 1550 nm to a tubular human tissue and inducing obtainment of an OCT image for a predetermined part of a tubular human tissue through location adjustment by near infrared ray emission by translational movement and rotational movement; and an optical fiber for treatment emitting laser of a predetermined wavelength to a lesion part of a tubular human tissue in a predetermined pattern, and stimulating the lesion part through location adjustment by laser emission by translational movement and rotational movement, wherein the optical fiber for treatment is at least one selected from one optically diffusing fiber emitting near infrared ray from an entire part of an outer circumference, and at least one side type optical fiber emitting near infrared ray only to a predetermined area limited in the lateral direction.

The hybrid optical medical equipment for both diagnosis and treatment of a tubular human tissue, wherein the human activating optical fiber module includes an optical fiber integrated coating body formed of a penetrating path for movably receiving the optical fiber for diagnosis and optical fiber for treatment independently, so that the optical fiber integrated coating body passes the inner passage of the probe.

The hybrid optical medical equipment for both diagnosis and treatment of a tubular human tissue, wherein the optically diffusing fiber is inserted into a balloon-shaped catheter passing through the inner passage of the probe and protruding to the front end of the probe, the balloon-shaped catheter having a balloon-shaped expansion tube arranged expandably at the end.

The hybrid optical medical equipment for both diagnosis and treatment of a tubular human tissue, wherein the human activating optical fiber module includes a single mode optical fiber which emits at least one selected from near infrared ray in a wavelength range of 800 to 1550 nm and laser of a predetermined wavelength to a tubular human tissue, controls the emission location by translational movement and rotational movement, and integrally performs inducement of obtainment of an OCT image for a predetermined part of the tubular human tissue and stimulation of a lesion part of the tubular human tissue.

The hybrid optical medical equipment for both diagnosis and treatment of a tubular human tissue, which further includes a camera having a photographing lens forming exposure towards the front end of the probe; and an optical source module for photography emitting visible rays through optical source bodies forming exposure towards the front end of the probe, thereby performing macroscopic monitoring of a tubular human tissue through a tubular human tissue image photographed by the camera and microscopic monitoring of the tubular human tissue through the OCT image, simultaneously.

The hybrid optical medical equipment for both diagnosis and treatment of a tubular human tissue, wherein the controller includes a controller for tissue diagnosis performing the operation control of the human activating optical fiber module for obtaining an OCT image of a human tissue; and a controller for laser treatment performing the operation control of the human activating optical fiber module for inducing photothermal treatment of the human tissue, and allowing Q-switched laser or pulse type laser in a wavelength of 532 nm, 980 nm, and 1470 nm to be emitted on a tubular human tissue having hemoglobin over a predetermined level.

The hybrid optical medical equipment for both diagnosis and treatment of a tubular human tissue, wherein the controller includes a controller for tissue diagnosis for performing the operation control of the human activating optical fiber module for obtaining an OCT image of a human tissue; and a controller for laser treatment performing the operation control of the human activating optical fiber module for inducing photothermal treatment of the human tissue, and allowing Q-switched frequency-doubled Nd:YAG 532 nm laser to be emitted on a tubular human tissue having blood vessel over a predetermined level.

The hybrid optical medical equipment for both diagnosis and treatment of a tubular human tissue, wherein the controller includes a controller for tissue diagnosis for performing the operation control of the human activating optical fiber module for obtaining an OCT image of a human tissue; and a controller for laser treatment performing the operation control of the human activating optical fiber module for inducing photothermal treatment of the human tissue, and allowing laser in a wavelength of 800 nm to be emitted on a tubular human tissue injected with a bio-dye material, indocyanine green.

A catheter-based laser treatment device, which includes: a catheter; a balloon having an inner space interconnected with the catheter, connected to an end of the catheter enabling expansion and contraction; a pressure controlling part inserting or discharging operation fluid to introduce the operation fluid into the balloon or discharge the operation fluid from the balloon through the catheter; an optical fiber inserted into the balloon penetrating through the catheter; a laser system transmitting laser through the optical fiber; a side type optical fiber inserted into the balloon penetrating through the catheter; and an imaging system transmitting and receiving light through the side type optical fiber to obtain an image of a tissue with the balloon inserted.

The catheter-based laser treatment device, wherein the optical fiber inserted into the balloon penetrating through the catheter is an optically diffusing fiber.

The catheter-based laser treatment device, wherein the pressure controlling part inserts or discharges the operation fluid at a pressure of 1 to 15 psi.

The catheter-based laser treatment device, wherein the pressure controlling part vibrates the balloon at a frequency of 1 to 100 Hz while maintaining a constant pressure.

The catheter-based laser treatment device, wherein the pressure controlling part generates a vibration wave, and the vibration wave is delivered to the balloon through the operation fluid.

The catheter-based laser treatment device, wherein at least one substance selected from the group consisting of an anti-inflammatory material, anti-infective material and anti-oxidation material having physiological compatibility is coated or impregnated on the surface of the balloon.

The catheter-based laser treatment device, wherein the pressure controlling part controls the insertion or discharge speed of the operation fluid so that the expansion and contraction speed of the balloon is 10 to 1000 μm/sec.

The catheter-based laser treatment device, wherein the pressure controlling part vibrates the balloon, simultaneously when the laser system emits laser to the tissue through the optical fiber.

An electromagnetic energy application device for tubular tissue stricture, which includes a catheter; a balloon catheter having an inner space interconnected with the catheter, connected to an end of the catheter enabling expansion and contraction; a pressure controlling part inserting or discharging operation fluid to introduce the operation fluid into the balloon catheter or discharge the operation fluid from the balloon catheter through the catheter; an optical fiber inserted into the balloon catheter penetrating through the catheter; a laser system transmitting laser through the optical fiber; and a location moving part withdrawing the balloon catheter.

The electromagnetic energy application device for tubular tissue stricture, wherein the optical fiber inserted into the balloon penetrating through the catheter is an optically diffusing fiber.

The electromagnetic energy application device for tubular tissue stricture, wherein the front end of the balloon catheter is formed in a sharp funnel shape, or the front and rear ends are symmetrically formed in a sharp funnel shape.

The electromagnetic energy application device for tubular tissue stricture, wherein the pressure controlling part inserts or discharges the operation fluid at a pressure of 1 to 15 psi.

The electromagnetic energy application device for tubular tissue stricture, wherein the pressure controlling part vibrates the balloon catheter at a frequency of 1 to 100 Hz while maintaining a constant pressure.

The electromagnetic energy application device for tubular tissue stricture, wherein the pressure controlling part generates a vibration wave, and the vibration wave is delivered to the balloon catheter through the operation fluid.

The electromagnetic energy application device for tubular tissue stricture, wherein the pressure controlling part controls the insertion or discharge speed of the operation fluid so that the expansion and contraction speed of the balloon catheter is 10 to 1000 μm/sec.

The electromagnetic energy application device for tubular tissue stricture, wherein the pressure controlling part vibrates the balloon catheter, simultaneously when the laser system emits laser to the tissue through the optical fiber.

### BRIEF DESCRIPTION OF THE DRAWINGS

FIG. 1 is a block diagram schematically illustrating a constitution of an apparatus for manufacturing an optically diffusing fiber probe according to an embodiment of the present invention;

FIG. 2 is an exemplary view illustrating a state of screen on which fabrication specification of an optically diffusing fiber probe according to an embodiment of the present invention;

FIG. 3 is an exemplary view illustrating a process for manufacturing an optically diffusing fiber probe;

FIG. 4 is a flow chart explaining a method for manufacturing an optically diffusing fiber probe according to the present invention;

FIG. 5 is an image showing scanning electron microscope (SEM) of an optical fiber fabricated according to an embodiment of the present invention;

FIG. 6 is a cross-section view illustrating an optically diffusing fiber probe according to various fabrication shapes according to an embodiment of the present invention;

FIG. 7 is an exemplary view illustrating laser emission in a plurality of directions by surface fabrication of an optical fiber according to an embodiment of the present invention;

FIG. 8 is an exemplary view illustrating optical energy distribution according to laser emission according to an embodiment of the present invention;

FIG. 9 and FIG. 10 are conceptual diagrams illustrating a basic constitution and operation of hybrid optical medical equipment for both diagnosis and treatment of a tubular human tissue according to the present invention;

FIG. 11 is a block diagram illustrating a constitution of hybrid optical medical equipment for both diagnosis and treatment of tubular human tissue according to an embodiment of the present invention;

FIGS. 12 (a) and (b) are views illustrating a constitution of a human activating optical fiber module according to an embodiment of the present invention which has optical fibers for diagnosis and treatment;

FIG. 13 (a) to (c) are views illustrating various arrangements of optical fibers for diagnosis and treatment forming a human activating optical fiber module according to an embodiment of the present invention;

FIGS. 14 (a) and (b) are views illustrating a balloon-type catheter applied to an optically diffusing fiber forming an optical fiber for treatment of a human activating optical fiber module according to an embodiment of the present invention;

FIG. 15 is a block diagram illustrating a constitution of hybrid optical medical equipment for both diagnosis and treatment of a tubular human tissue according to another embodiment of the present invention;

FIG. 16 is a view illustrating a constitution of a human activating optical fiber module according to another embodiment of the present invention with a single mode optical fiber;

FIG. 17 is a view illustrating a front end of a probe of hybrid optical medical equipment for both diagnosis and treatment of a tubular human tissue according to an embodiment of the present invention;

FIG. 18 is a view illustrating a schematic shape of hybrid optical medical equipment for both diagnosis and treatment of a tubular human tissue according to an embodiment of the present invention;

FIG. 19 is a view explaining laser emission and drug delivery process to tissue by a catheter-based laser treatment device of the present invention;

FIG. 20 is a view illustrating an observation of tissue conjugated through a catheter-based laser treatment device of the present invention;

FIG. 21 is an exemplary view illustrating a state of proceeding with vascular stricture through a balloon catheter according to the present invention;

FIG. 22 is an exemplary view illustrating a process of proceeding with optical treatment by inserting an optical fiber into the balloon catheter according to the present invention;

FIG. 23 is an exemplary view illustrating a state of continuously delivering a uniform heat to a vessel wall by adjusting a pressure inside a balloon catheter as the blood vessel is adsorbed according to the present invention;

FIG. 24 is an exemplary view proceeding with a targeted treatment by expanding as much as a unique diameter of a blood vessel through monitoring according to the present invention;

FIG. 25 is an exemplary view illustrating a treatment state of an entire blood vessel through motion control after clarifying a treatment range through a balloon catheter according to the present invention to proceed with a partial treatment;

FIG. 26 is an image illustrating an optically diffusing fiber fabricated for the treatment of endometrium;

FIG. 27 (a) illustrates a constitution of experiment for photocoagulation through an optical fiber, and FIG. 27 (b) illustrates a distribution of light strength of an optically diffusing fiber covered with a cap measured every 5 mm;

FIG. 28 illustrates a spatial distribution of photon through an optical simulation comparing an optically diffusing fiber with a capped optically diffusing fiber at various distances of 1, 5 and 10 mm;

FIG. 29 illustrates a progress of tissue coagulation according to emission time induced by a laser;

FIG. 30 (a) is quantitative data of tissue coagulation depth according to emission time (according to a direction of radial form), and FIG. 30 (b) illustrates coagulation at tissue surface and distribution of heat which spreads to the side;

FIG. 31 (a) illustrates a combination of an optically diffusing fiber with a balloon catheter for endometrial coagulation, and FIG. 31 (b) illustrates a thermal reaction of uterus tissue of goat after 2 hours of 30-seconds coagulation using a prototype;

FIG. 32 is an image showing histological tissue stained with H&E after laser treatment;

FIG. 33 is a view illustrating a test on uterus tissue of human after in vivo experiment using a prototype; and

FIG. 34 illustrates a novel type of optically diffusing equipment designed to solve the problems of geometrical characteristics of uterus and movement of optical fiber tip.

### DETAILED DESCRIPTION OF THE EMBODIMENTS

The present invention provides an optically diffusing fiber capable of emitting light in a plurality of directions and a method for manufacturing the same, and a probe including the optically diffusing fiber for treating tubular tissue diseases or solid cancers (thyroid cancer, breast cancer kidney cancer, etc.) When using the optically diffusing fiber according to the present invention, electromagnetic energy could be constantly emitted in a plurality of directions, thereby treating a broader range of diseases in a safe and efficient way.

Also, the present invention may be applied to photothermal treatment or photodynamic therapy through insertion into an inner tissue of a human body by using an optically diffusing fiber capable of emitting light in a plurality of directions. Additionally, the optically diffusing fiber may be used for treating thyroid cancer, breast cancer, prostate cancer, kidney cancer, bladder cancer, brain tumor, inner uterine wall, localized liver cancer, skin cancer, cancer tissue, coagulation of inner tissue, removal of fat, etc.

Also, according to hybrid optical medical equipment for both diagnosis and treatment of a tubular human tissue according to the present invention, obtainment of an OCT image for the tubular human tissue such as bronchus, blood vessel and ureter, and induction of photothermal treatment of human tissue by laser may be integrally performed through a single probe, thereby increasing efficiency of lesion diagnosis of tubular human tissue and induction of treatment. Also, the OCT image for the human tissue may be monitored in real time before and after performing the induction of photothermal treatment of human tissue, thereby efficiently performing diagnosis for lesion tissue and induction of treatment while minimizing damage on the human tissue. Especially, according to hybrid optical medical equipment for both diagnosis and treatment of a tubular human tissue of the present invention, diagnosis for various respiratory diseases such as asthma and induction of treatment may be promoted. Furthermore, the equipment may be applied to various surgery fields, so the usability thereof could be increased.

Additionally, a catheter-based laser treatment device according to the present invention has effects of preventing tracheal stricture from recurring after surgery, and minimizing complications such as inflammation, injection, etc. which may occur during recovery. Also, the catheter-based laser treatment device performs treatment while monitoring in real time a part to be treated during treatment, thereby minimizing damage on tissue caused by the photothermal treatment.

According to the present invention, the use of various balloon catheters with geometric shape may minimize hemorrhage through blood vessels before or during treatment by using expansion of the balloon catheters, and induce vascular stricture without contraction of the balloon catheters.

In addition, according to the present invention, the use of a fixed shape of balloon catheter allows automatic induction of deflation of catheter according to vasoconstriction during laser treatment.

The present invention relates to an optically diffusing fiber probe, a method for manufacturing the same, and an application thereof. More specifically, the present invention relates to an optically diffusing fiber probe capable of emitting light in a plurality of directions and a method for manufacturing the same, and hybrid optical medical equipment for both diagnosis and treatment of a tubular human tissue, a catheter-based laser treatment device, and an electromagnetic energy application device for tubular tissue stricture, including the optically diffusing fiber probe.

1. An Optically Diffusing Fiber Probe and a Method for Manufacturing the Same

The first aspect of the present invention relates to an optically diffusing fiber, an optical fiber probe for treating a tissue disease or a solid cancer including the optically diffusing fiber, and a method for manufacturing the same.

The optically diffusing fiber according to the present invention has a fabrication length L of a tissue treatment section required for laser treatment; a tapering angle α and an end diameter d within the fabrication length capable of uniformly delivering optical energy; a fabrication angle β and fabrication part interval w capable of varying optical energy distribution delivered; and a height p of an optically diffusing surface fabricated to vary a diffusion range of optical energy.

The optically diffusing fiber probe for treating a tissue disease or a solid cancer according to the present invention includes an optically diffusing fiber as described above.

Additionally, a method for manufacturing an optically diffusing fiber probe for treating a tissue disease or a solid cancer according to the present invention includes the steps of (a) inputting fabrication values including an optically diffusing range according to a disease part to be treated, energy distribution, optical fiber fabrication length L, tapering angle α, end diameter d, fabrication angle β, fabrication part interval w, and height p of optically diffusing surface for manufacturing a suitable optical fiber for treatment length, etc.; (b) outputting a fabrication control signal through a fabrication controlling part; (c) fabricating a side surface and a front end of an optical fiber by moving the optical fiber in the rotational direction and front and back direction according to the fabrication control signal; (d) delivering optical energy to an optical fiber; (e) measuring optical energy delivered to the side surface and front end of an optical fiber through a side surface optical sensor and a front optical sensor; and (f) determining whether to go through additional fabrication and polishing by comparing the energy distribution of the measured strength and pre-stored optical fiber.

According to the present invention, as to the fabrication length L, the initial fabrication location of the optical fiber is determined together with an entire fabrication length in consideration of a translational stage. The tapering angle α and end diameter d of the optical fiber is determined by simultaneously or independently controlling the translational speed, rotational speed, power of fabrication energy source (0.1 W to 50 W), and area of energy source of the optical fiber. The fabrication angle β and fabrication part interval w are determined by simultaneously or independently controlling the translational speed and rotational speed of the optical fiber. The height p of the optically diffusing surface is determined by controlling the rotational speed of the optical fiber, power of fabrication energy source (0.1 W to 50 W) and area of energy source.

In the method for manufacturing the optically diffusing fiber for treating a tissue disease or a solid cancer according to the first aspect of the present invention, the step (f) may further include the step of conducting a feedback for precise fabrication when determined to go through an additional fabrication, and the precise fabrication minutely controls fabrication delivery speed, rotational speed, and fabrication energy.

The step (a) may further include the step (a-1) of controlling the fabrication length L of the optical fiber in consideration of the tissue treatment section required for laser treatment, and the step (a-1) determines an initial fabrication location of the optical fiber with an overall fabrication length in consideration of a translational stage.

The step (a) may further include the step (a-2) of determining the tapering angle α and end diameter d of the optical fiber so that light of the optical energy is uniformly delivered through the optical fiber, and the step (a-2) determines the tapering angle α and end diameter d of the optical fiber by simultaneously or independently controlling the translational speed, rotational speed, power of fabrication energy source (0.1 W to 50 W), and area of energy source of the optical fiber.

The step (a) may further include the step (a-3) of determining a fabrication angle β and a fabrication part interval w to vary the optical energy distribution delivered through the optical fiber, and the step (a-3) determines the fabrication angle β and fabrication part interval w by simultaneously or independently controlling the translational speed and rotational speed of the optical fiber.

The step (a) may further include the step (a-4) of determining the height p of the optically diffusing surface to vary the diffusion range of optical energy light through the optical fiber, and the step (a-4) determines the height p of the optically diffusing surface by controlling the rotational speed of the optical fiber, power of fabrication energy source (0.1 W to 50 W) and area of energy source.

Hereinafter, embodiments of the optically diffusing fiber, the optical fiber probe for treating a tissue disease or a solid cancer including the optically diffusing fiber, and a method for manufacturing the same according to the first aspect of the present invention will be explained in detail with reference to the accompanying drawings. First, in adding reference numerals to constitutional elements of each drawing, the same constitutional element is to have the same reference numeral, if possible, even though the constitutional element is illustrated in another drawing. Additionally, when it is determined that detailed explanation on related well-known constitution or function may make the gist of the present invention unclear, the detailed explanation thereon will be omitted. Additionally, hereinafter, preferred embodiments of the present invention will be explained, but it is of course that the technical idea of the present invention is not limited thereto, but can be carried out by a skilled person in the art.

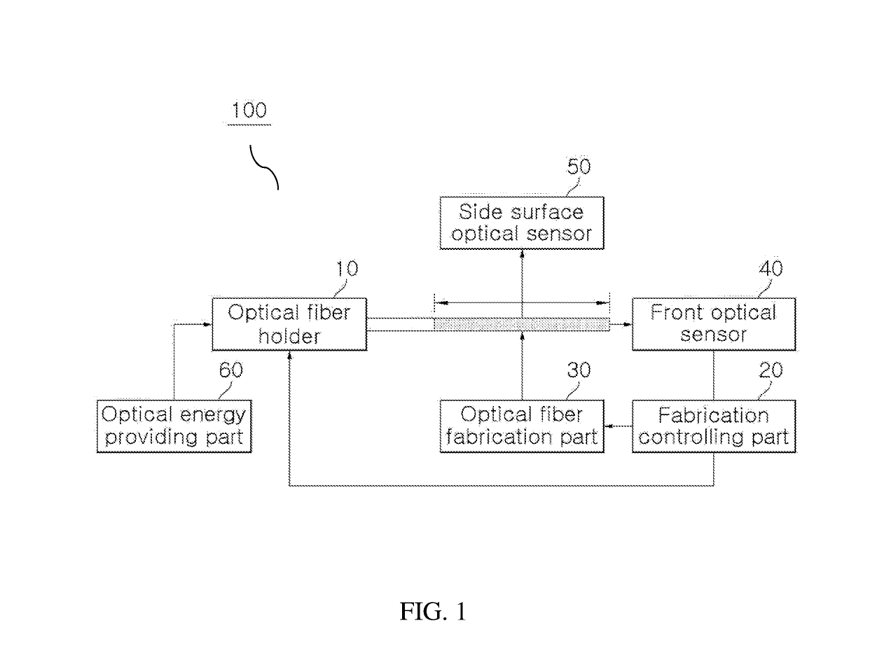

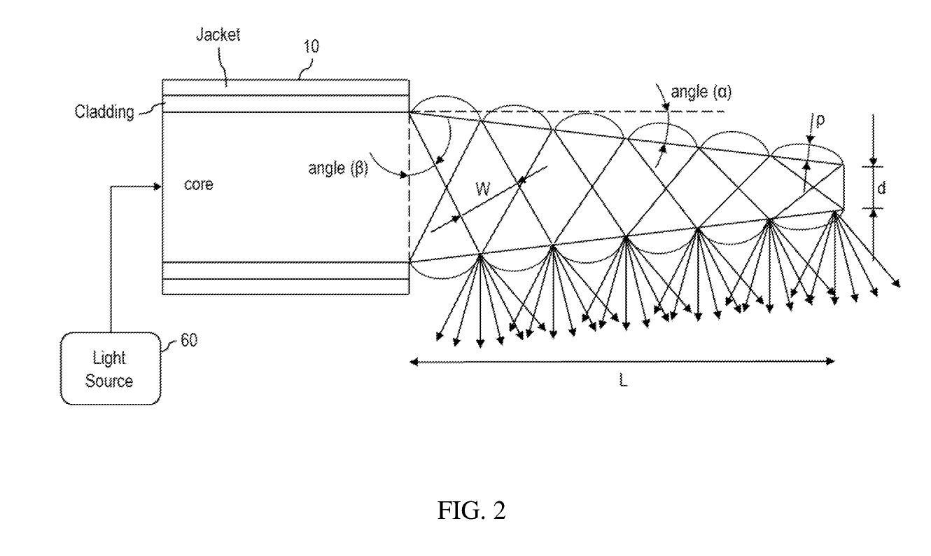

FIG. 1 is a block diagram schematically illustrating a constitution of an apparatus for manufacturing an optically diffusing fiber probe according to the present invention, and FIG. 2 is an exemplary view illustrating a state of screen on which fabrication specification of the optically diffusing fiber probe according to an embodiment of the present invention.

As illustrated in FIG. 1 and FIG. 2, the optically diffusing fiber according to a preferred embodiment of the present invention is manufactured to emit light in a plurality of directions unlike the conventional optical fiber which is manufactured to emit light in a fixed direction (front or side). Accordingly, the optically diffusing fiber according to the present invention can constantly emit electromagnetic energy in a plurality of directions to a tubular tissue disease or a solid cancer (thyroid cancer, breast cancer, kidney cancer, etc.), and can be used for treating a broader range of diseases in a safe and efficient way.

Here, an optical fiber generally includes a core providing a path through which light is delivered and a cladding surrounding the core. In the present invention, both a single-mode optical fiber and a multi-mode optical fiber may be used according to transmission type of light.

An optically diffusing fiber probe according to the present invention may be manufactured by using an optical fiber probe manufacturing device 100 including an optical fiber holder 10, a fabrication controlling part 20, an optical fiber fabrication part 30, a side surface optical sensor 50, a front optical sensor 40, and an optical providing part 60.

The optical fiber holder 10 installs an optical fiber, which is the object to be fabricated. A rotational motor, which is not illustrated, is driven according to a control signal of the fabrication controlling part 20 to rotate the optical fiber.

The fabrication controlling part 20 outputs a fabrication control signal which controls the optical fiber holder 10 and the optical fiber fabrication part 30 based on predetermined fabrication values in consideration of optically diffusing range of light, energy distribution, treatment length, etc., for the optical fiber to be fabricated.

The optical fiber fabrication part 30 fabricates and polishes an optical fiber, which is the object to be fabricated, installed on the optical fiber holder 10. The optical fiber fabrication part fabricates and polishes the optical fiber by driving the rotational motor, which is not illustrated, according to the fabrication control signal to move towards side surface and front surface of the optical fiber.

The optical energy providing part 60 provides optical energy to an optical fiber fabricated and is delivered through the optical fiber holder 10. The side surface optical sensor 50 and the front optical sensor 40 are installed at the side surface or front end of the optical fiber to measure a strength of optical energy, to confirm whether the optical energy delivered from the optical energy providing part 60 is smoothly emitted to the side surface and front end of the optical fiber.

The fabrication controlling part 20 compares the strength of optical energy measured from the side surface optical sensor 50 and front optical sensor 40 with the predetermined energy distribution of the optical fiber to determine whether to add fabrication and polishing.

The fabrication controlling part 20 applies and controls the fabrication control signal, which minutely controls the fabrication delivery speed, rotational speed, fabrication energy, etc. for optimizing the optical fiber, to the optical fiber holder 10 and the optical fiber fabrication part 30.

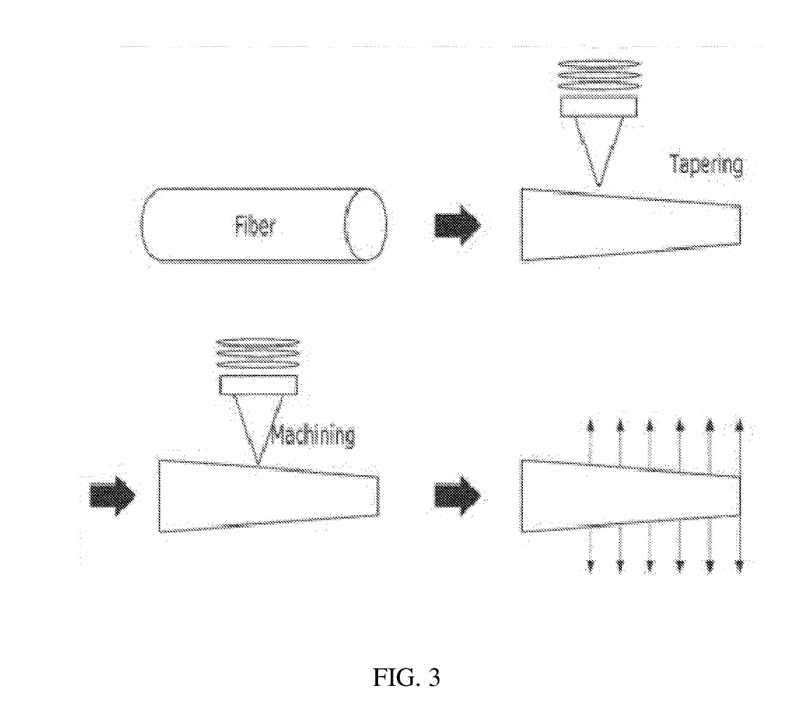

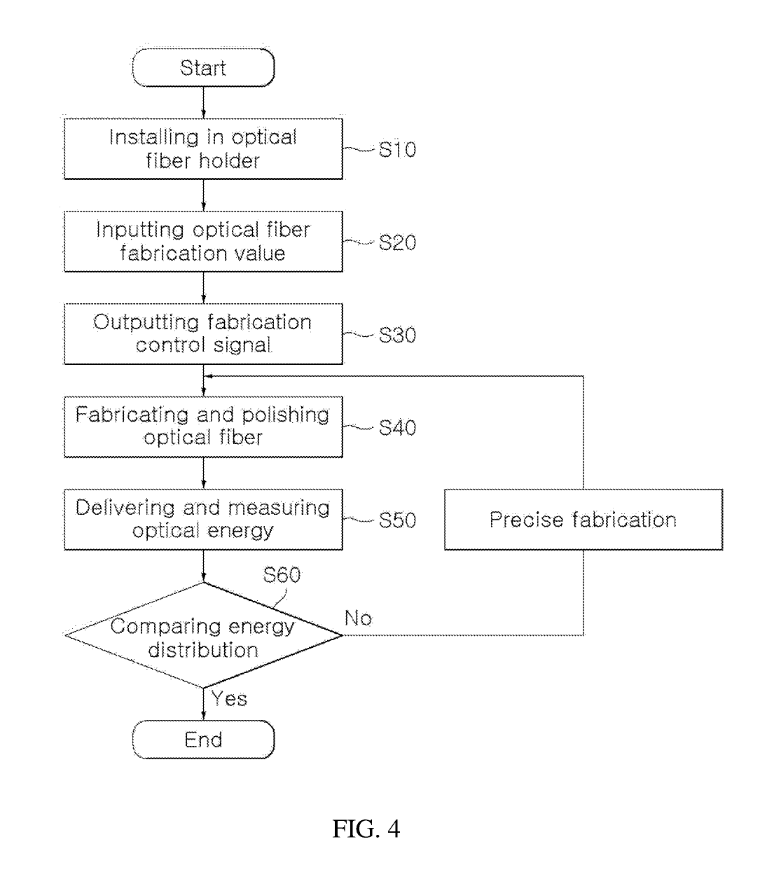

FIG. 3 is an exemplary view illustrating a process for manufacturing an optically diffusing fiber probe, and FIG. 4 is a flow chart explaining a method for manufacturing an optically diffusing fiber probe according to the present invention.

As illustrated in FIG. 3 and FIG. 4, the method for manufacturing the optically diffusing fiber probe according to the preferred embodiment of the present invention is to manufacture an optical fiber probe capable of emitting light in a plurality of directions unlike the conventional optical fiber, which emits light only in a fixed direction (front or side). The method fabricates a tapering which makes an end smaller while removing a surface through a rotational movement of the optical fiber, fabricates the side by translational/rotational movement to have a surface of optical fiber embossed, and delivers optical energy light to the fabricated optical fiber so that the delivered optical energy light could be diffused into the side.

To this end, the method for manufacturing the optically diffusing fiber probe according to the present invention installs an optical fiber to be fabricated on the optical fiber holder 10 (S10), and inputs fabrication values through a monitor and a key input part, which are not illustrated, in consideration of light diffusing range, energy distribution, treatment length, etc., for the optical fiber to be fabricated (S20).

When the input of the fabrication values is completed, the fabrication controlling part 20 outputs a fabrication control signal which controls the optical fiber holder 10 and the optical fiber fabrication part 30 (S30), and the optical fiber holder 10 drives the rotational motor (not illustrated) according to the fabrication control signal to rotate the optical fiber installed on the optical fiber holder 10 (S40).

The optical fiber fabrication part 30 moves in the front and back direction according to the fabrication control signal, and fabricates and polishes the side surface and front end of the optical fiber installed on the optical fiber holder 10 (S40).

When the fabrication and polishing of the optical fiber is completed by the optical fiber fabrication part 30, the optical energy is delivered to the fabricated optical fiber using the optical energy providing part 60, and whether the optical energy provided from the optical energy providing part 60 is delivered to the side surface and front end of the optical fiber is measured by the side surface optical sensor 50 and front optical sensor 40 (S50).

The fabrication controlling part 20 compares the strength measured by the side surface optical sensor 50 and front optical sensor 40 with the pre-stored energy distribution of the optical fiber to determine whether to go through additional fabrication and polishing (S60).

In the step S60, when it is determined to go through additional fabrication and polishing by the fabrication controlling part 20, a feedback process for precise fabrication is performed (S70). Here, as to the precise fabrication, the fabrication controlling part 20 applies the fabrication control signal, which minutely controls again fabrication delivery speed, rotational speed, fabrication energy, etc., to the optical fiber holder 10 and optical fiber fabrication part 30. Thus, the step S30 is repetitively performed.

Meanwhile, a process of inputting fabrication values in consideration of light diffusing range for the optical fiber to be fabricated, energy distribution, treatment length, etc. (S20) is as below.

First, the optical fiber fabrication length L is adjusted in consideration of tissue treatment section required for laser treatment. Here, an initial fabrication location of the optical fiber is determined with an entire fabrication length in consideration of a translational stage.

Thereafter, a tapering angle α and a diameter d of the optical fiber end are determined so that light of optical energy can be uniformly delivered through the optical fiber. For example, the tapering angle α and diameter d of the optical fiber end are determined by simultaneously or independently controlling the translational speed, rotational speed, power of fabrication energy source (0.1 W to 50 W), and area of energy source of the optical fiber.

Also, in order to vary the optical energy distribution delivered through the optical fiber, a fabrication angle β and a fabrication part interval w are determined. For example, the fabrication angle β and fabrication part interval w are determined by simultaneously or independently controlling the translational speed and rotational speed of the optical fiber.

Also, in order to vary the light diffusing range of optical energy through the optical fiber, a height p of the optically diffusing surface fabricated is determined. For example, the height p of the optically diffusing surface is determined by controlling the rotational speed of the optical fiber, power of fabrication energy source (0.1 W to 50 W) and area of energy source.

Thereafter, whether light of optical energy provided from the optical energy providing part (light source, 60) through the optical fiber side surface fabrication is uniformly delivered to all directions through the side surface and front end of the optical fiber.

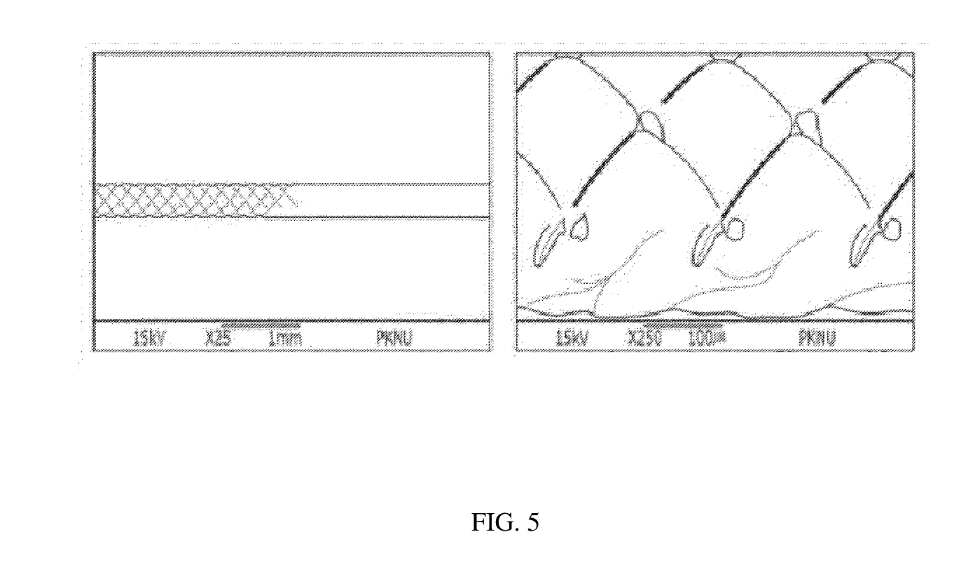

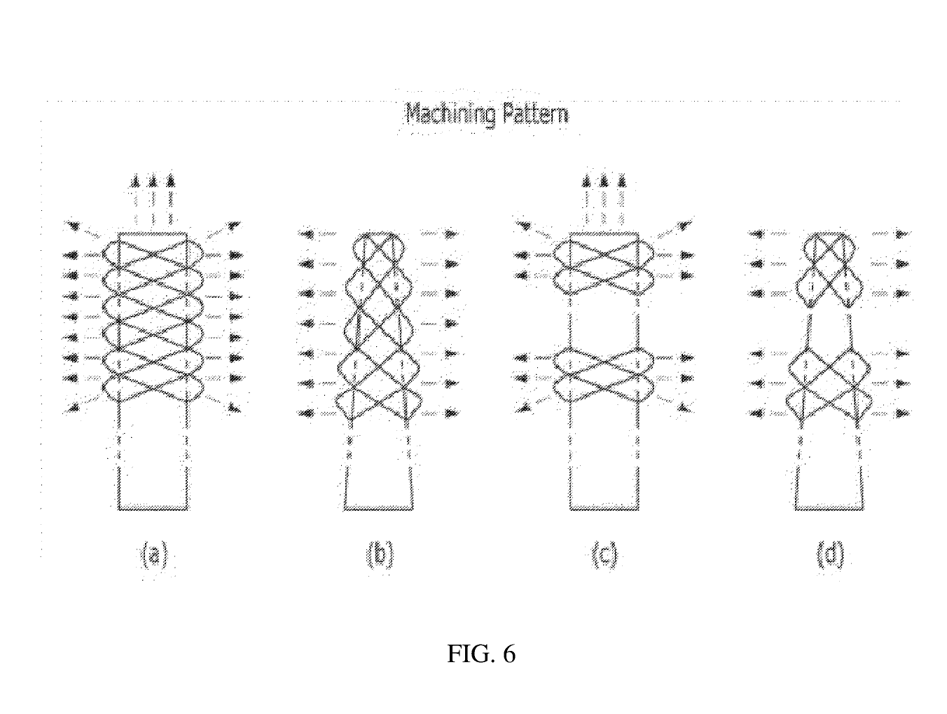

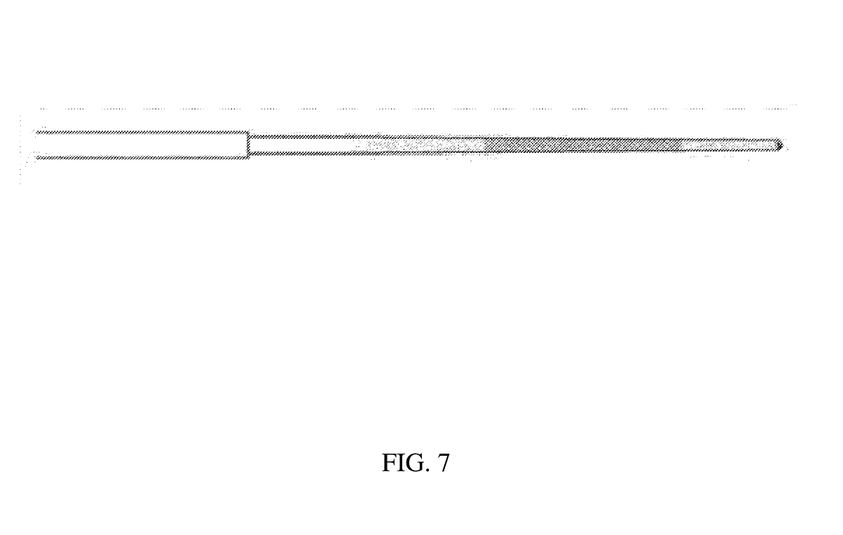

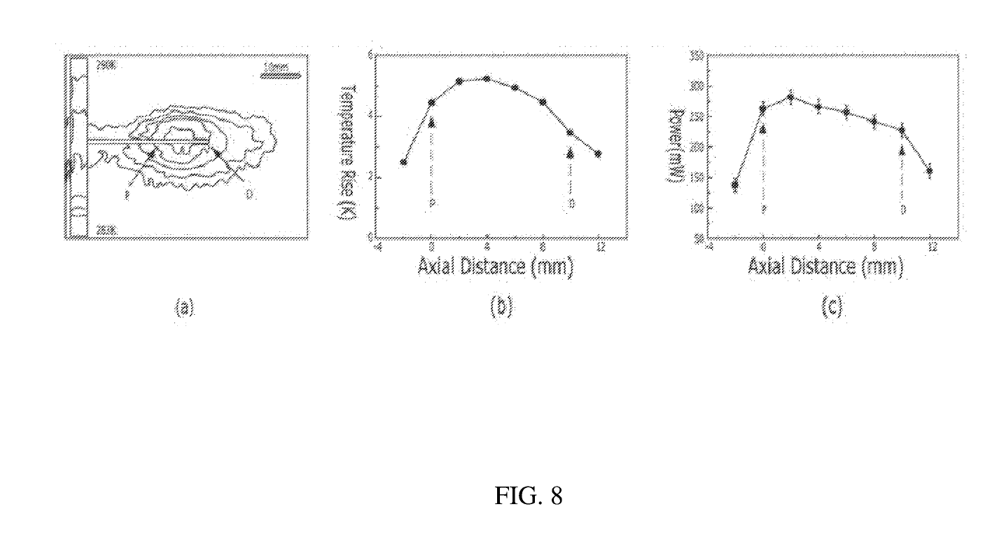

FIG. 5 is an image showing scanning electron microscope (SEM) of an optical fiber fabricated according to an embodiment of the present invention, FIG. 6 is a cross-section view illustrating an optically diffusing fiber probe according to various fabrication shapes according to an embodiment of the present invention, FIG. 7 is an exemplary view illustrating laser emission in a plurality of directions by surface fabrication of an optical fiber according to an embodiment of the present invention, and FIG. 8 is an exemplary view illustrating optical energy distribution according to laser emission according to an embodiment of the present invention.

As illustrated in FIG. 5 to FIG. 8, the optically diffusing fiber probe according to the present invention is an optically diffusing fiber probe capable of emitting light in a plurality of directions. For example, as the optically diffusing fiber probe constantly emits electromagnetic energy in a plurality of directions to a tubular tissue disease or a solid cancer (thyroid cancer, breast cancer, kidney cancer, etc.), thereby treating a broader range of diseases in a safe and efficient way. The optically diffusing fiber probe fabricates and modifies a side surface and a surface of the optical fiber in consideration of various fabrication conditions (fabrication angle, cladding removal rate, fabrication depth, size of optically diffusing surface, length of optically diffusing part, interval of optically diffusing surface, etc.).

Here, the fabrication angle of the optical fiber surface is controlled to between 0° and 90° according to light diffusing range of optical energy. At an angle of 0°, a partial optical emission is possible radially (ring type), and at an angle of 90°, a partial optical emission is possible axially (it is possible to induce emission in all directions at an angle between 0° and 90°).

In order to determine the optical energy distribution, the optically diffusing fiber probe adjusts a size of optically diffusing surface (i.e., diameter) formed in a side surface of the optical fiber to between 0.01 mm and 0.4 mm, and adjusts the fabrication depth, interval of optically diffusing surface, power of fabrication energy source, area of energy source, etc., to determine the size of optically diffusing surface.

As the surface size of the optically diffusing fiber probe is smaller, a higher density of energy distribution is possible. As the size thereof is greater, a relatively lower density of optical energy distribution is possible. The fabrication length of the optical fiber can be determined according to the size of optical energy tissue treatment (i.e., 0.5 to 5 cm).

For uniformly distributing electromagnetic energy, the optically diffusing fiber probe fabricates tapering of the optical fiber, performs side surface energy distribution focusing inducement at the end according to the angle (15 to 75°) of the tapering, and adjusts the fabrication translational speed to within a range of 0.5 to 10 mm/s for tapering fabrication.

By tapering the diameter of the end of the optical fiber between 0.05 to 0.2 mm, loss at the end of the optical energy can be reduced within 5%. Additionally, by tapering the diameter of the end of the optical fiber between 0.2 to 0.8 mm, 10 to 50% of entire optical energy can be emitted at the end in front direction.

The optically diffusing fiber probe determines the degree of fabrication of the optical fiber core and cladding according to desired electromagnetic energy distribution, controls the fabrication rotational speed to within 60 to 500 rpm according to the cladding removal range, and simultaneously or independently controls the fabrication energy to 0.1 W to 50 W and fabricate the energy.

Here, for partial and selective optical diffusion (deep fabrication depth: 0.05 to 0.5 mm), slow speed (10 to 200 rpm) is applied, and for broad optical diffusion (swallow surface fabrication: 0.01 to 0.05 mm), fast speed (200 to 1000 rpm) is applied.

Also, the optically diffusing fiber probe determines distribution and directional properties of optical energy in a desired direction according to the side surface and surface fabrication processing of the optical fiber. Here, the electromagnetic energy distribution includes Flat-top, Gaussian, Left-skewed, Right-skewed, Fractional, Diffuse, Radial, etc.

The directional properties of the electromagnetic energy include Front, Fractional, Cylindrical, Spherical, etc. Additionally, the fabrication interval is controlled to between 0.05 and 0.8 mm for controlling distribution form of optical energy, and the fabrication translational speed is controlled to between 0.5 and 10 mm/s for uniform energy distribution according to an axis of optical fiber.

The optically diffusing fiber probe uses non-contact mechanical or electromagnetic energy source for optical fiber surface fabrication. Here, the electromagnetic energy source includes femto second, picosecond, ultraviolet laser, arc discharge, etc. The fabrication power is adjusted to within 0.01 to 50 W to induce a change in the fabrication degree of the optical fiber surface. Additionally, the fabrication surface of the optical fiber can be polished using the energy source after fabricating the optical fiber for continuous optical diffusion.

The optically diffusing fiber probe determines a method for processing a side surface and a surface of the optical fiber according to desired distribution of electromagnetic energy. The side surface energy distribution form may be implemented with flat-top or Gaussian, by making the size of optically diffusing surface greater (diameter of 0.1 to 0.3 mm) at an end and a starting end of the optical fiber, and making the size of optically diffusing surface smaller (diameter of 0.05 to 0.09 mm) at a center portion.

The optically diffusing fiber probe uses an energy sensor to identify energy distribution of fabricated optical fiber, and carries out fabrication optimization. Here, when the length of optical fiber is 1 cm or greater, the fabrication size and fabrication depth per section of optical fiber are changed to induce uniform energy distribution in the lateral direction. Additionally, the fabrication size and depth are changed every length within 15 to 40% of the entire optical fiber to constantly maintain energy distribution at an end and a starting end of the optical fiber.

The optically diffusing fiber probe is inserted into the tissue disease, and may induce photothermal coagulation, photodynamic therapy, or tissue removal for desired tissue. Additionally, the optically diffusing fiber can be used for treating thyroid cancer, breast cancer, prostate cancer, kidney cancer, bladder cancer, brain tumor, inner uterine wall, localized liver cancer, skin cancer, cancer tissue, coagulation of inner tissue, removal of fat, etc.

As described above, the present invention manufactures an optical fiber probe capable of emitting light in a plurality of directions unlike the conventional optical fiber, which simply emits light in a fixed direction (front or side) to constantly emit electromagnetic energy in a plurality of directions to a tubular tissue disease or a solid cancer (thyroid cancer, breast cancer, kidney cancer, etc.), thereby treating a broader range of diseases in a safe and efficient way.

2. Hybrid Optical Medical Equipment for Both Diagnosis and Treatment of a Tubular Human Tissue.

The second aspect of the present invention relates to hybrid optical medical equipment for both diagnosis and treatment of a tubular human tissue using a probe including an optically diffusing fiber.

The hybrid optical medical equipment for both diagnosis and treatment of tubular human tissue according to the present invention includes a probe moving by being inserted in a tubular human tissue; a human activating optical fiber module protruding to the front end of the probe by passing an inner passage of the probe, the human activating optical fiber module performing any one selected from obtaining an optical coherence tomography (OCT) image of a tubular human tissue through infrared light emission of a predetermined wavelength area and inducing tubular human tissue photothermal treatment through laser emission; a controller connected to the human activating optical fiber module, performing the operation control of the human activating optical fiber module for obtaining an OCT image of human tissue and for inducing human tissue photothermal treatment; and an OCT image output device connected to the controller, outputting an OCT image obtained from the human activating optical fiber module.

According to one embodiment of the hybrid optical medical equipment for both diagnosis and treatment of a tubular human tissue according to the present invention, the human activating optical fiber module may include an optical fiber for diagnosis inducing obtainment of an OCT image for a predetermined part of a tubular human tissue through location adjustment by near infrared ray emission by translational movement and rotational movement, the human activating optical fiber module emitting near infrared ray in a wavelength range of 800 to 1550 nm to a tubular human tissue; and an optical fiber for treatment emitting laser of a predetermined wavelength to a lesion part of a tubular human tissue in a predetermined pattern, and stimulating the lesion part through location adjustment by laser emission by translational movement and rotational movement.

The optical fiber for treatment may be at least one selected from one optically diffusing fiber emitting near infrared ray from an entire part of an outer circumference, and at least one side type optical fiber emitting near infrared ray only to a predetermined area limited in the lateral direction. The optically diffusing fiber may be inserted into a balloon-shaped catheter passing through the inner passage of the probe and protruding to the front end of the probe, and the balloon-shaped catheter may have a balloon-shaped expansion tube arranged expandably at the end.

It is preferable that the human activating optical fiber module includes an optical fiber integrated coating body formed of a penetrating path for movably receiving the optical fiber for diagnosis and optical fiber for treatment independently, so that the optical fiber integrated coating body passes the inner passage of the probe.

According to another embodiment of the present invention, the human activating optical fiber module includes a single mode optical fiber which emits at least one selected from near infrared ray in a wavelength range of 800 to 1550 nm and laser of a predetermined wavelength to a tubular human tissue, controls the emission location by translational movement and rotational movement, and integrally performs inducement of obtainment of an OCT image for a predetermined part of the tubular human tissue and stimulation of a lesion part of the tubular human tissue.

The hybrid optical medical equipment for both diagnosis and treatment of a tubular human tissue according to another embodiment of the present invention further includes a camera having a photographing lens forming exposure towards the front end of the probe; and an optical source module for photography emitting visible rays through optical source bodies forming exposure towards the front end of the probe, thereby performing macroscopic monitoring of a tubular human tissue through a tubular human tissue image photographed by the camera and microscopic monitoring of the tubular human tissue through the OCT image, simultaneously.

According to another embodiment of the present invention, the controller includes a controller for tissue diagnosis performing the operation control of the human activating optical fiber module for obtaining an OCT image of a human tissue; and a controller for laser treatment performing the operation control of the human activating optical fiber module for inducing photothermal treatment of the human tissue, the controller for laser treatment allowing Q-switched laser or pulse type laser in a wavelength of 300 to 3000 nm to be emitted on a tubular human tissue having hemoglobin over a predetermined level, Q-switched frequency-doubled Nd:YAG 532 nm laser to be emitted on a tubular human tissue having blood vessel over a predetermined level, or laser in a wavelength of 800 nm to be emitted on a tubular human tissue injected with a bio-dye material, indocyanine green.

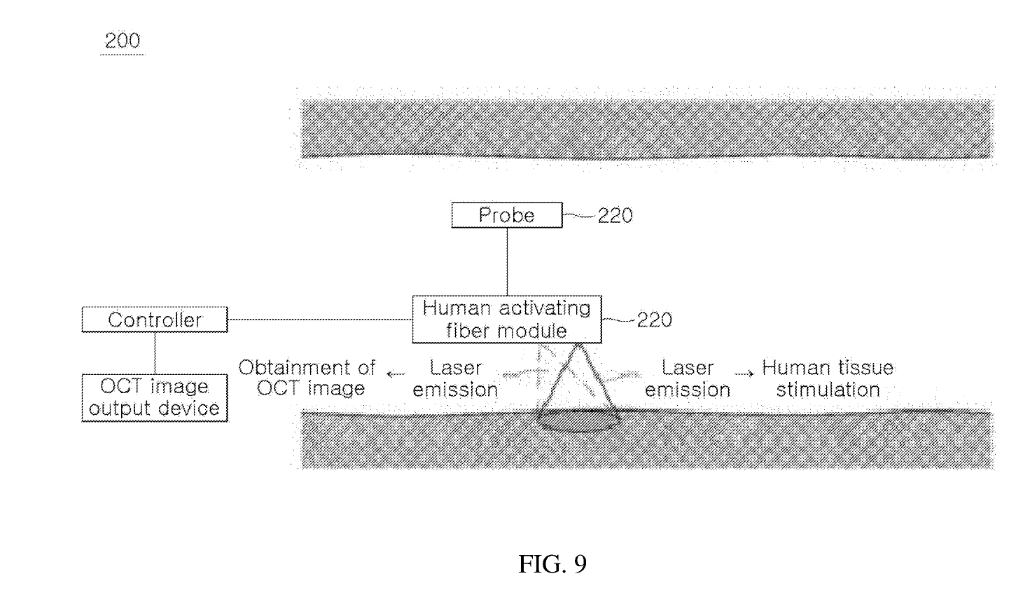

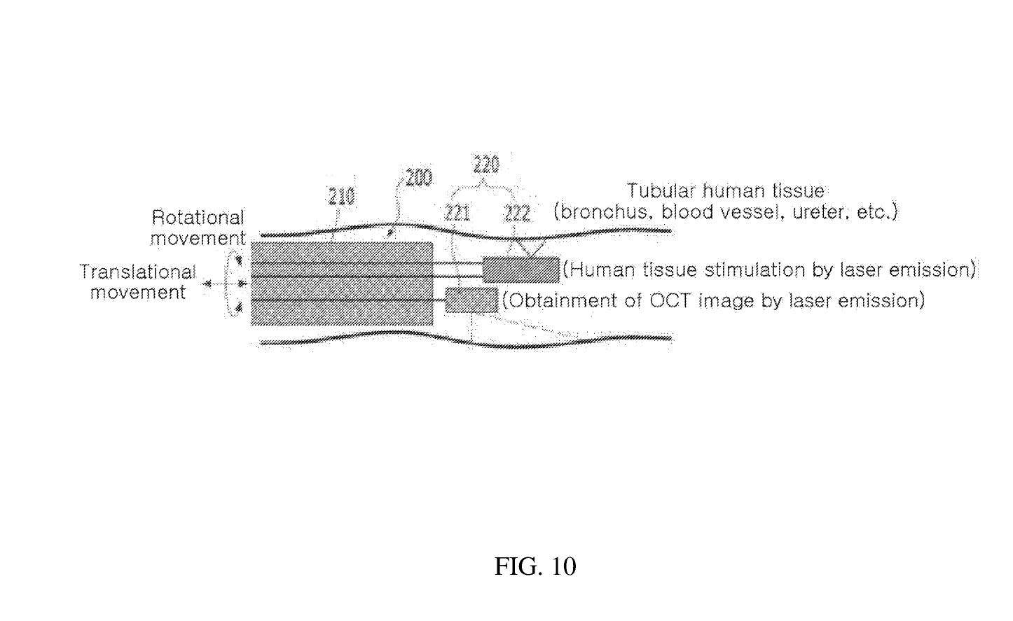

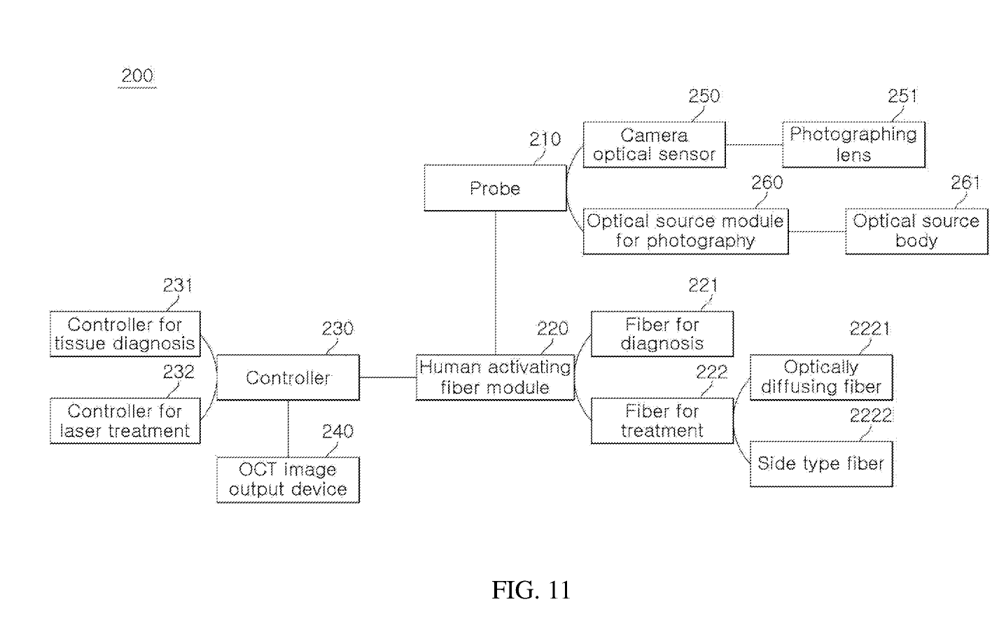

Hereinafter, embodiments of the present invention will be explained in detail with reference to FIG. 9 to FIG. 18. Meanwhile, in the drawings and detailed description, any illustration and explanation on the constitution and operation which a skilled person in the art can easily understand from general probe, optical coherence tomography (OCT), optical fiber, infrared ray, laser, catheter, etc. are briefly described or omitted. Especially, in illustration of drawings and detailed description, the detailed explanation and illustration on specific technical constitution and operation of elements which are not directly related with the technical characteristics of the present invention are omitted. Only the technical feature related with the present invention will be briefly illustrated or explained.

As illustrated in FIG. 9 to FIG. 12, hybrid optical medical equipment 200 for both diagnosis and treatment of a tubular human tissue according to an embodiment of the present invention includes a probe 210, a human activating optical fiber module 220, a controller 230, an OCT image output device 240, a camera 250, and an optical source module for photography 260, and integrally performs the OCT image monitoring and laser stimulation for the tubular human tissue.

The probe 210 moves by being inserted into a tubular human tissue such as bronchus, blood vessel, and ureter. As the probe 210, a probe included in an endoscope or a bronchoscope may be used.

The human activating optical fiber module 220 protrudes to the front end of the probe 210 by passing an inner passage 211 of the probe 210. This human activating optical fiber module 220 obtains an OCT image of a tubular human tissue through infrared ray emission of a predetermined wavelength area, and induces photothermal treatment of the tubular human tissue through laser emission. Here, the OCT image obtainment of the tubular human tissue through infrared ray emission is performed during and before and after inducement of photothermal treatment of the tubular human tissue through laser emission. By obtaining the OCT image of the tubular human tissue, a change in smooth muscle under an epithelial cell may be observed, and the degree of treatment of a lesion part and the degree of thermal damage of the tubular human tissue may be observed in real time.

The human activating optical fiber module 220 according to the embodiment of the present invention includes an optical fiber for diagnosis 221 and an optical fiber for treatment 222. Independent inducement of the movements (translational movement and rotational movement) of the optical fiber for diagnosis 221 and the optical fiber for treatment 222 allows real-time diagnosis and treatment inducement for the tubular human tissue.

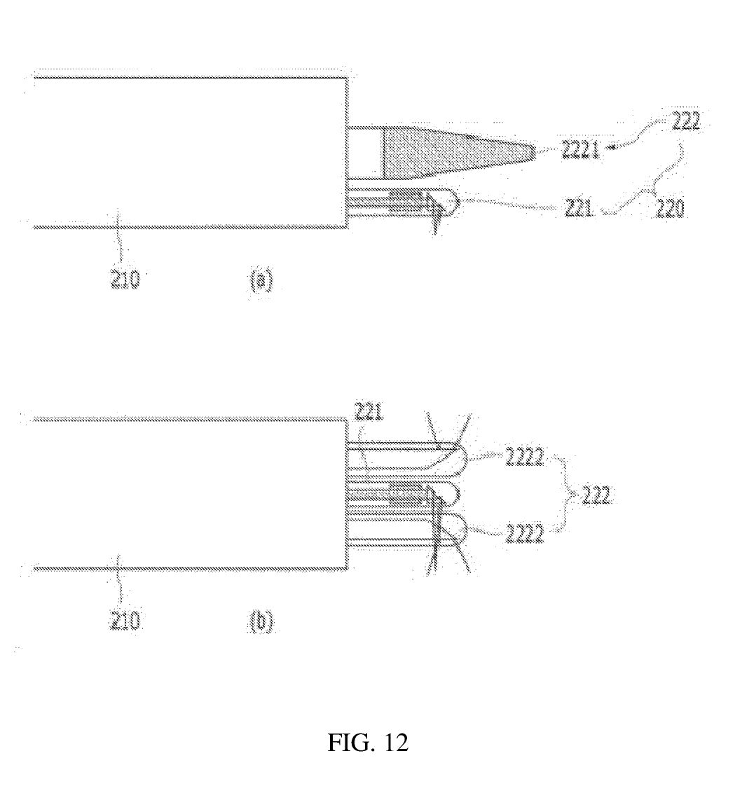

The optical fiber for diagnosis 221 emits near infrared ray in a wavelength range of 800 to 1550 nm to the tubular human tissue; and obtains the OCT image for a predetermined part of the tubular human tissue through location adjustment by near infrared ray emission by translational movement and rotational movement. As such, the optical fiber for diagnosis 221 is configured to emit near infrared ray to a predetermined area limited in the lateral direction as shown in FIGS. 12 (a) and (b) to obtain the OCT image of the predetermined area to which near infrared ray is emitted.

The optical fiber for treatment 222 emits laser of a predetermined wavelength to a lesion part of a tubular human tissue in a predetermined pattern, and stimulates the lesion part through location adjustment by laser emission by translational movement and rotational movement. At least one optical fiber for treatment 222 is selected from at least one optically diffusing fiber 2221 and at least one side type optical fiber 2222, and the constitution of the optical fiber for treatment 222 is determined according to a structure of lesion part of the tubular human tissue and treatment thickness required.

The optically diffusing fiber 2221, which is an optical fiber allowing near infrared ray to be emitted from an entire part of an outer circumference surface, is used when photothermal coagulation of overall tubular human tissue is required. The optically diffusing fiber 2221 has short optical penetrating depth properties and constant laser energy distribution properties, and thus may allow limited and uniform treatment inducement for a human tissue.

The side type optical fiber 2222, which is an optical fiber allowing near infrared ray to be emitted only to a predetermined area limited in the lateral direction, is used when photothermal coagulation of a part of tubular human tissue is required. The side type optical fiber 2222 may deliver high laser energy, and thus it is used when incision of human tissue or coagulation of relatively thick human tissue is required.

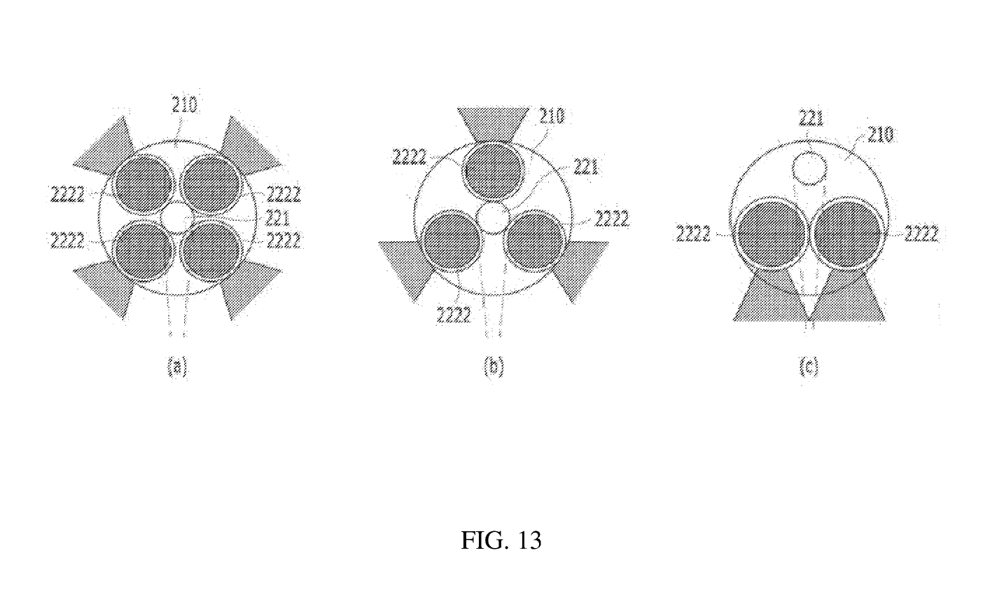

According to an embodiment of the present invention, the human activating optical fiber module 220 may include the optical fiber for diagnosis 221, and the optical fiber for treatment 222 including the optically diffusing fiber 2221, as illustrated in FIG. 12 (a). Additionally, as illustrated in FIG. 12 (b), the human activating optical fiber module 220 may include the optical fiber for diagnosis 221, and the optical fiber for treatment 222 consisting of a plurality of side type optical fibers 2222. Here, when the human activating optical fiber module 220 according to the embodiment of the present invention has the optical fiber for treatment 222 consisting of a plurality of side type optical fibers 2222, four side type optical fibers 2222 may be disposed with an angle of 90° along the circumference of the optical fiber for diagnosis 221 disposed in the center of a probe 210 as illustrated in FIG. 13 (a). Additionally, as illustrated in FIG. 13 (b), three side type optical fibers 2222 may be disposed with an angle of 120° along the circumference of the optical fiber for diagnosis 221 disposed in the center of the probe 210. Also, two side type optical fibers 2222 may be disposed at an end of the probe 210 space apart from the optical fiber for diagnosis 221 disposed at another end of the probe 210. However, the constitution of the optical fiber for treatment 222 consisting of a plurality of side type optical fibers 2222 is not limited thereto. The constitution of the optical fiber for treatment 222 consisting of a plurality of side type optical fibers 2222 is determined according to the number of side type optical fiber 2222, which may promote structure of a lesion part of the tubular human tissue, required treatment thickness, minimization of incidence rate of complications and increase in treatment efficiency at the same time.

The optically diffusing fiber 2221 forming the optical fiber for treatment 222 may pass an inner passage 211 of the probe 210 to be inserted into the inside of a balloon-shaped catheter 225 protruding toward the front end of the probe 210. By the balloon-shaped catheter 225, swift and safe human tissue treatment inducement may be possible while inducing uniform temperature increase in all directions. Here, the balloon-shaped catheter 225 has balloon-shaped expansion tubes 2251 and 2251′ disposed expandably to an end. The balloon-shaped expansion tubes 2251 and 2251′ may be expanded through saline solution, thereby modifying the balloon-shaped expansion tubes 2251 and 2251′ in accordance with structural characteristics of the tubular human tissue.

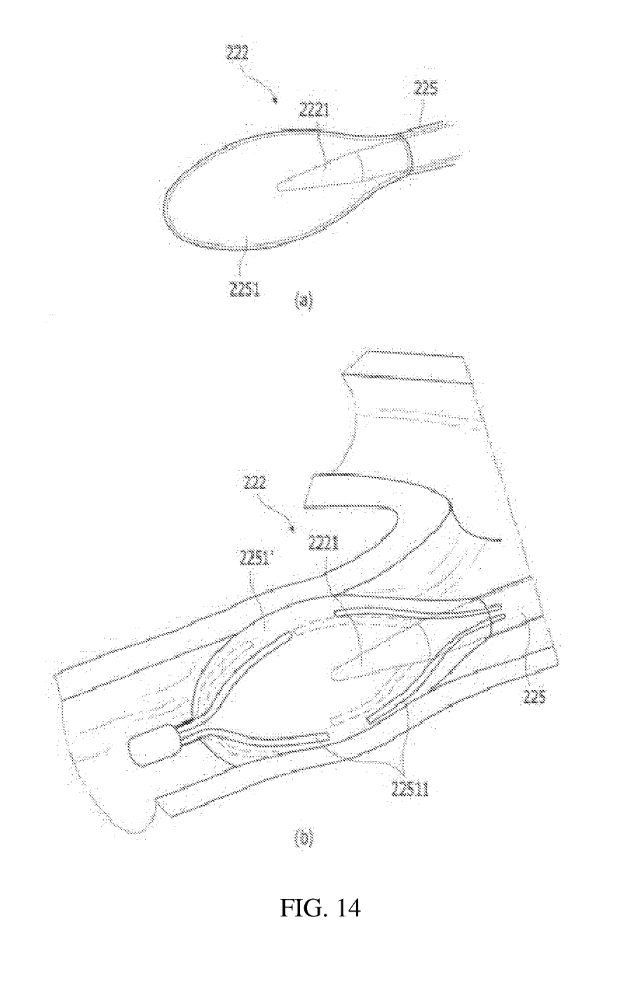

The balloon-shaped expansion tubes 2251 and 2251′ have an inner space interconnected with the inner passage of the balloon-shaped catheter 225. According to the embodiment of the present invention, the balloon-shaped catheter 225 has the balloon-shaped expansion tube 2251 formed to be extended from an end as illustrated in FIG. 14 (a) so that the end of the balloon-shaped catheter 225 could be disposed in the inner space of the balloon-shaped expansion tube 2251. Or, the balloon-shaped catheter 225 has the balloon-shaped expansion tube 2251′ formed in a predetermined area at an end as illustrated in FIG. 14 (b) so that the end of the balloon-shaped catheter 225 could be formed by passing through the inner space of the balloon-shaped expansion tube 2251′. The balloon-shaped expansion tube 2251′ illustrated in FIG. 14 (b) is supported by a plurality of ribs 22511 each of which is radially formed at both sides of the end of the balloon-shaped catheter 225 in the longitudinal direction. Accordingly, the change in shape of the balloon-shaped expansion tube 2251′ which is expanded and shrunk may be limited by the plurality of ribs 22511, and thereby a surface of human tissue to which the balloon-shaped expansion tube 2251′ is adhered may be protected, and expansion and contraction of the balloon-shaped expansion tube 2251′ may be stably performed.

Additionally, a glass cap is fitted into an end of the optically diffusing fiber 2221 so that the end of the optical fiber could be protected, and laser emitted from the optically diffusing fiber 2221 is uniformly diffused to all directions without directional properties.

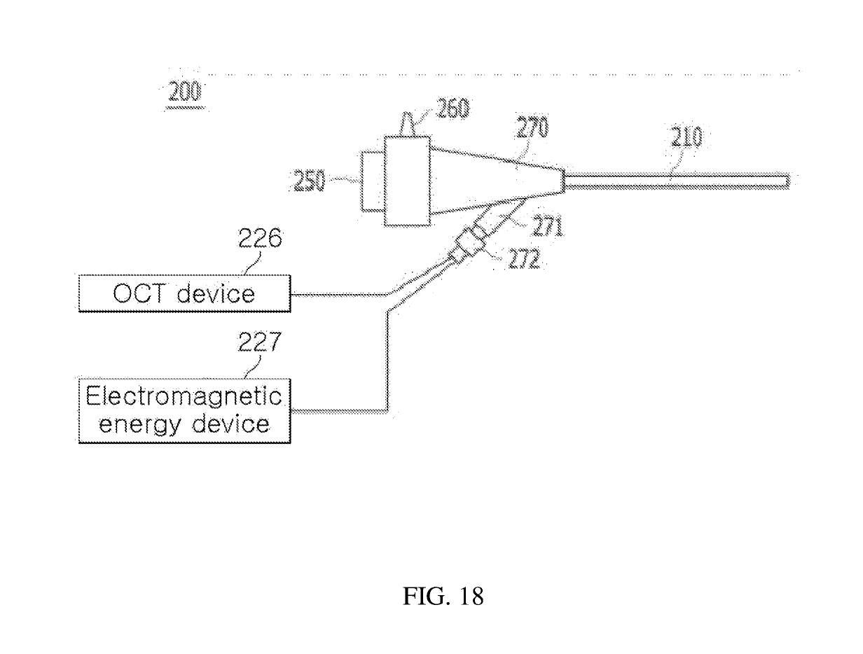

According to an embodiment of the present invention, in the human activating optical fiber module 220, the optical fiber for diagnosis 221 and the optical fiber for treatment 222 are connected to an OCT device 226 and electromagnetic energy device 227 as illustrated in FIG. 18 to obtain an OCT image by near infrared ray emission and induce photothermal treatment of human tissue by laser emission. As the optical fiber for diagnosis 221 and the optical fiber for treatment 222 independently perform translational movement and rotational movement by a pair of small motors 272 which are independently installed on a channel entry 271 of an optical medical equipment body end 270 to induce real-time diagnosis and treatment for tubular human tissue. As the small motor 272, a piezo-actuator may be used.

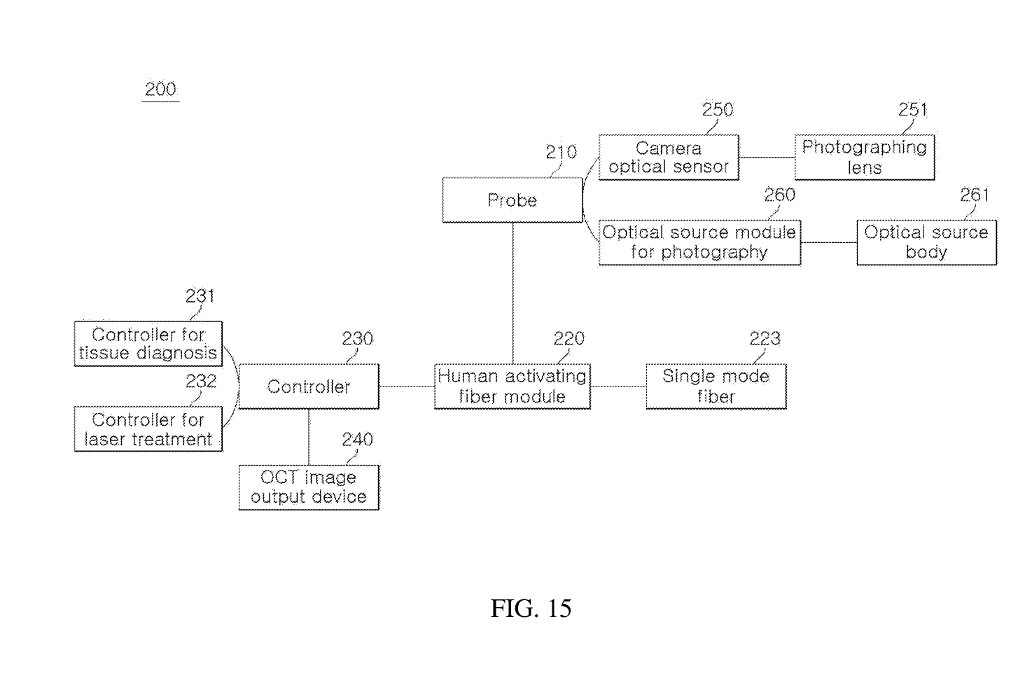

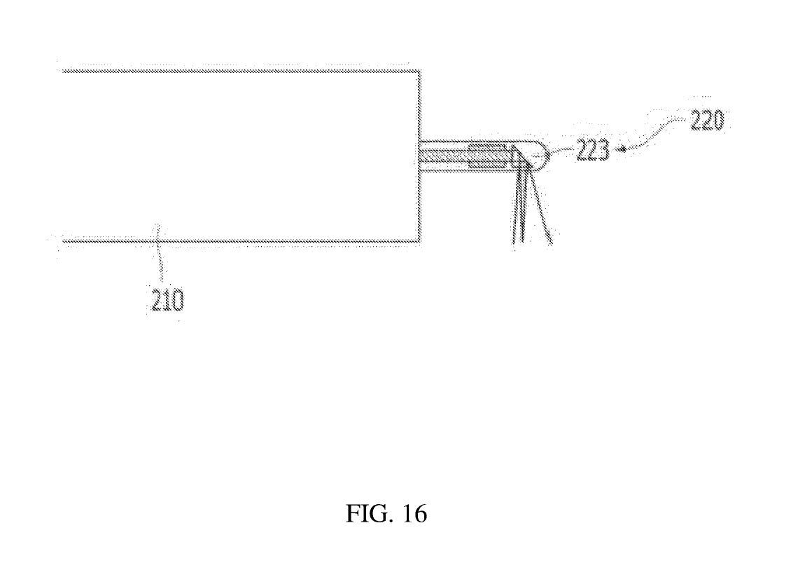

The human activating optical fiber module 220 according to another embodiment of the present invention has one single mode optical fiber 223 as illustrated in FIG. 15 and FIG. 16. The single mode optical fiber 223 is used when a diameter of a tubular human tissue is 1 mm or less, which is very small, and it couples and applies near infrared ray wavelength for obtaining the OCT image and laser wavelength for inducing photothermal treatment of human tissue to the optical fiber in the shape of side type optical fiber. Especially, the single mode optical fiber 223 may be effectively applied to peripheral blood vessels to which micro-treatment is required or treatment of bronchus end.

The single mode optical fiber 223 selectively emits near infrared ray in the wavelength of 800 to 1550 nm or laser with a predetermined wavelength to a tubular human tissue. The single mode optical fiber 223 may integrally perform the OCT image obtainment for a predetermined part of the tubular human tissue and stimulation for a lesion part of the tubular human tissue, while controlling emission location through the translation movement and rotational movement.

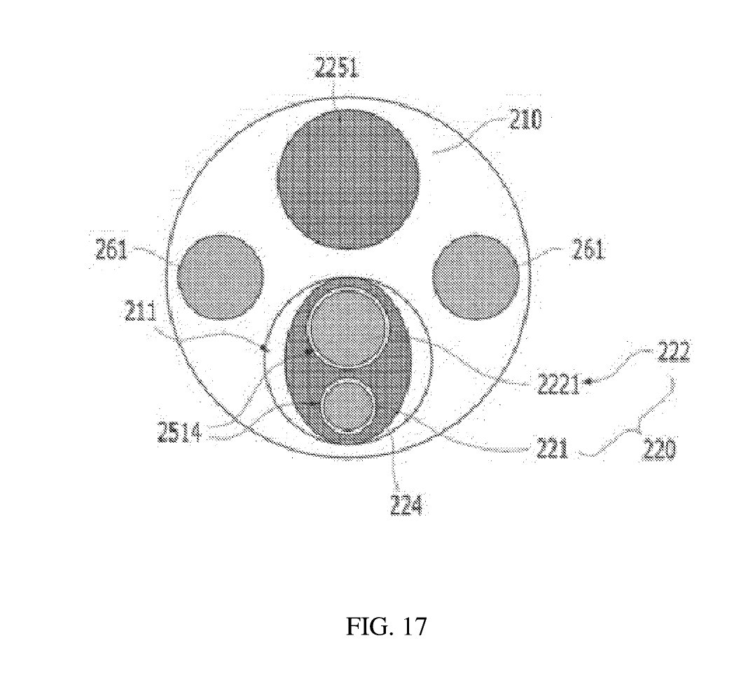

Meanwhile, according to an embodiment of the present invention, as illustrated in FIG. 17, the human activating optical fiber module 220 includes an optical fiber integrated coating body 224 formed of a penetrating path 2514 for movably receiving the optical fiber for diagnosis 221 and optical fiber for treatment 222 independently, so that the optical fiber integrated coating body 224 passes the inner passage 211 of the probe 210.

A controller 230 is connected to the human activating optical fiber module 220, and performs the operation control of the human activating optical fiber module for obtaining an OCT image of human tissue and the operation control of the human activating optical fiber module for inducing photothermal treatment of human tissue. For this, the controller 230 includes a controller for tissue diagnosis 231 and a controller for laser treatment 232. The controller for tissue diagnosis 231 controls the operation of human activating optical fiber module for obtaining the OCT image of human tissue. The controller for laser treatment 232 controls the operation of the human activating optical fiber module for inducing photothermal treatment of human tissue.

The controller for laser treatment 232 may allow Q-switched laser or pulse type laser in a wavelength of 300 to 3000 nm to be emitted on a tubular human tissue having hemoglobin over a predetermined level through the human activating optical fiber module 220. Additionally, the controller for laser treatment 232 may allow Q-switched frequency-doubled Nd:YAG 532 nm laser to be emitted on tubular human tissue having blood vessel over a predetermined level through the human activating optical fiber module 220. By minimum invasion from the pulse type laser in a short wavelength, the lesion part of tubular human tissue may be removed.

When it is necessary to clearly distinguish the lesion part of tubular human tissue, indocyanine green, which is a bio-dye material, or dye inducing optical absorption reaction may be injected into a tubular human tissue so that treatment efficiency could be increased when inducing laser photothermal treatment. In this case, the controller for laser treatment 232 allows the laser in the wavelength of 800 nm to be emitted to the tubular human tissue to which indocyanine green, bio-dye material, through the human activating optical fiber module 220.

An OCT image output device 240 is connected to the controller 230 to output an OCT image obtained from the human activating optical fiber module 220.

A camera 250 has a photographing lens 251 forming exposure towards the front end of the probe 210 as illustrated in FIG. 17 and FIG. 18, and it takes an image of front end of the probe 210.

An optical source module for photography 260 emits visible rays through optical source bodies 261 forming exposure towards the front end of the probe 210, as illustrated in FIG. 17 and FIG. 18. By the optical source module for photography 260, the camera 250 can take the image of front end of the probe 210.

Here, the hybrid optical medical equipment 200 for both diagnosis and treatment of tubular human tissue according to an embodiment of the present invention may perform macroscopic monitoring of a tubular human tissue through a tubular human tissue image photographed by the camera 250 and microscopic monitoring of the tubular human tissue through the OCT image output by the OCT image output device 240.

The hybrid optical medical equipment 200 for both diagnosis and treatment of tubular human tissue according to an embodiment of the present invention configured as above induces photothermal treatment for tubular human tissues such as bronchus, blood vessel, and ureter through the human activating optical fiber module 220 installed by passing through the inside of the single probe 210, and performs real-time monitoring of OCT image for human tissue during and before and after the inducement of photothermal treatment of human tissue by the same human activating optical fiber module 200, thereby integrally performing diagnosis and treatment inducement for a lesion part while minimizing damage on human tissue. Additionally, the hybrid optical medical equipment 200 for both diagnosis and treatment of tubular human tissue according to the embodiment of the present invention includes the optical fiber for diagnosis 221 and the optical fiber for treatment 222. As the translation movement and rotational movement of the optical fiber for diagnosis 221 and the optical fiber for treatment 222 are independently induced to allow real-time diagnosis and treatment inducement for tubular human tissue. By performing macroscopic monitoring of tubular human tissue through the camera 250 and optical source module for photography 260 and microscopic monitoring of tubular human tissue through the obtained OCT image simultaneously, during and before and after inducement of laser photothermal treatment by the optically diffusing fiber 221, side type optical fiber 222 disposed in a predetermined pattern, single mode optical fiber 223, etc., the present invention allows precise diagnosis and treatment for an initial lesion within tubular tissues which was difficult to be treated with the conventional technology.

3. A Catheter-Based Laser Treatment Device

The third aspect of the present invention relates to a catheter-based laser treatment device, which includes a catheter; a balloon having an inner space interconnected with the catheter, connected to an end of the catheter enabling expansion and contraction; a pressure controlling part inserting or discharging operation fluid to introduce the operation fluid into the balloon or discharge the operation fluid from the balloon through the catheter; an optical fiber inserted into the balloon penetrating through the catheter; a laser system transmitting laser through the optical fiber; a side type optical fiber inserted into the balloon penetrating through the catheter; and an imaging system transmitting and receiving light through the side type optical fiber to obtain an image of a tissue with the balloon inserted.

The pressure controlling part inserts or discharges the operation fluid at a pressure of 1 to 15 psi.

Additionally, the pressure controlling part vibrates the balloon at a frequency of 1 to 100 Hz while maintaining a constant pressure.

Also, the pressure controlling part generates a vibration wave, and the vibration wave is delivered to the balloon through the operation fluid.

Also, at least one substance selected from the group consisting of an anti-inflammatory material, anti-infective material and anti-oxidation material having physiological compatibility is coated or impregnated on the surface of the balloon.

Also, the pressure controlling part controls the insertion or discharge speed of the operation fluid so that the expansion and contraction speed of the balloon is 10 to 1000 μm/sec.

Also, the pressure controlling part vibrates the balloon, simultaneously when the laser system emits laser to the tissue through the optical fiber.

Hereinafter, preferred embodiments of the catheter-based laser treatment device according to the present invention will be explained in detail with reference to the drawings attached.

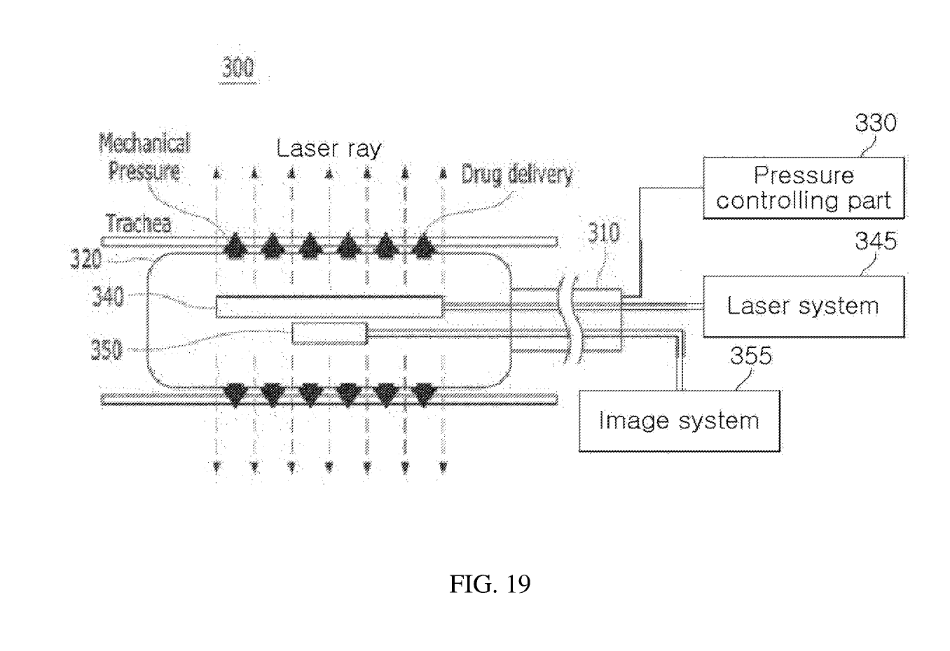

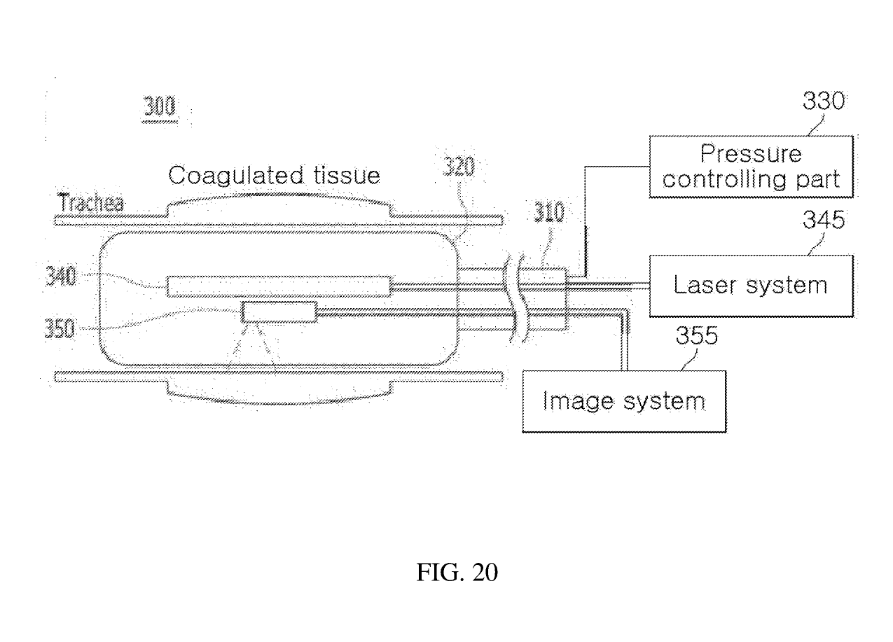

FIG. 19 is a view explaining laser emission and drug delivery process to tissue by a catheter-based laser treatment device according to an embodiment of the present invention, and FIG. 20 is a view illustrating an observation of tissue conjugated through a catheter-based laser treatment device according to an embodiment of the present invention.

Hereinafter, the catheter-based laser treatment device 300 according to preferred embodiment of the present invention will be explained with reference to FIG. 19 and FIG. 20.

The catheter-based laser treatment device 300 according to a preferred embodiment of the present invention includes a catheter 310, a balloon 320, a pressure controlling part 330, an optical fiber 340, a laser system 345, a side type optical fiber 350, and an imaging system 355.

The catheter 310 is formed in a tubular shape and inserted into the body, and the optical fiber 340 and side type optical fiber 350 are inserted through the inner penetrating path.

The balloon 320 has an inner space interconnected with the catheter 310, and connected to an end of the catheter 310 enabling expansion and contraction.

The balloon 320 is formed of a material from which laser ray emitted through the optical fiber 340 is penetrated into the tissue to be treated.

Additionally, an anti-inflammatory material, anti-infective material and anti-oxidation material having physiological compatibility is coated or impregnated on the surface of the balloon 320.

The balloon 320 where the drug is coated or impregnated on its surface is inserted into trachea and is expanded so that the drug could be delivered to the tissue to be treated while contacting a part to be treated in trachea.

As the drug is delivered to the part to be treated along with photothermal treatment by the laser ray, the complications such as inflammation, infection, etc. of the tissue to be treated would be minimized.

The drug coated on the surface of the balloon 320 is not limited to an anti-inflammatory anti-infective, anti-oxidant material. Any material may be coated or impregnated if the material is useful for the treatment.

The pressure controlling part 330 inserts or discharges the operation fluid to expand or contract the balloon 320 through the catheter 310 to introduce the operation fluid into the balloon 320 or discharge the operation fluid from the balloon 320.

In this case, the operation fluid, for example, consists of fluid which is harmless to human body even if it is inserted into trachea like air or saline solution. Additionally, the pressure controlling part 330 and catheter 310 are directly connected, or interconnected through an additional conduit so that the operation fluid could be flowed through the conduit.

The pressure controlling part 330 may be implemented with means such as a pump inserting or discharging the operation fluid. Preferably, the pressure controlling part may be implemented with an electronic pump capable of precisely controlling the amount of insertion or discharge of the fluid according to a predetermined speed.

Specifically, the pressure controlling part 330 controls the speed of insertion or discharge of the operation fluid so that the speed of expansion and contraction of the balloon 320 could be 10 to 1000 μm/sec.

Additionally, the pressure controlling part 330 is capable of expanding or contracting the balloon 320 by inserting or discharging the operation fluid with a pressure of 1 to 15 psi.

The pressure controlling part 330 allows the balloon 320 to be expanded or contracted with various speeds and pressures. Additionally, the balloon 320 gives or releases the pressure to the tissue coagulated by the laser ray so that the corresponding tissue could be expanded or permanently modified.

At this time, when the speed of expansion and contraction of the balloon 320 is less than 10 μm/sec, the expansion and contraction speed are very slow, and thus it would be difficult to induce modification of tissue within a given time. When the speed of expansion and contraction exceeds 1000 μm/sec, the speed is excessively fast, and thus it would not be easy to control the pressure of the balloon 320 and the tissue could be damaged by a sudden expansion pressure.

Also, when the pressure expanding and contracting the balloon 320 is less than 1 psi, the pressure is so low, and thus it would be difficult to induce modification. It is possible to sufficiently modify the tissue in the trachea under the pressure below 15 psi, so the pressure exceeding 15 psi is unnecessary. The pressure exceeding 15 psi may excessively press the tissue so the tissue could be damaged.

Furthermore, the pressure controlling part 330 is configured to vibrate the balloon 320 at a frequency of 1 to 100 Hz while maintaining a constant pressure. The pressure controlling part 330 causes periodical expansion and contraction of coagulated tissue through the balloon 320 so that the trachea may permanently and easily control the size modified and modification rate.

For this, the pressure controlling part 330 may include a means for generating a vibration wave (not illustrated) generating a vibration wave, and the vibration wave generated by this is delivered to the balloon 320 through the operation fluid.

The pressure controlling part 330 is capable of repetitively inserting and discharging a small amount of operation fluid to the balloon 320 according to a fixed time interval in order to vibrate the balloon 320 with a fixed interval.

The optical fiber 340 is passed through the catheter 310. Additionally, one end thereof is inserted into the inside of the balloon 320, and the laser system 345 transmitting the laser through the optical fiber 340 is disposed at another end of the optical fiber 340.

The optical fiber 340 is formed of the optically diffusing fiber. Additionally, the probe or glass cap for diffusing or condensing laser ray with a proper form according to the necessity may be included in the one end of the optical fiber 340.

The laser system 345 is connected to the optical fiber 340 to supply laser ray, and the laser system 345 controls the wavelength of laser ray, emission strength and emission interval according to the properties of tissue to be treated.

The pulsed laser and continuous wave laser (cw laser) may be used as laser supplied to the optical fiber 340 by the laser system 345. As the wavelength of laser, a visible ray wavelength, a near infrared ray wavelength, a medium infrared wavelength, a far infrared ray wavelength, etc. may be applied.

In this case, the laser system 345 may include a laser diode capable of modulating an output signal in order to control emission strength of laser ray through which the degree of penetration of laser ray into the tissue to be treated and temperature may be precisely controlled.

Meanwhile, the side type optical fiber 350 passes through the catheter 310 like the optical fiber 340, and one end thereof is inserted into the inside of the balloon 320. Another end of the side surface optical fiber 350 is connected to the imaging system 355, and the imaging system 355 transmits and receives light or optical signal through the side surface optical fiber 350 to obtain an image of tissue of a part to which the balloon 320 is inserted.

Here, the imaging system 355 may be implemented an image photographing device such as an optical coherence tomography (OCT) device, a photoacoustic tomography device, a polarization imaging device, etc.

Additionally, the side type optical fiber 350 may be coupled to the optical fiber 340 inside the catheter 310 or balloon 320, and this allows the surface type optical fiber 350 to perform translational and rotational movement along with the optical fiber 340.

As the side type optical fiber 350 is coupled to the optical fiber 340 to be moved and rotated together, the laser emitted through the optical fiber 340 is emitted to the tissue, and thus a photocoagulation process of tissue could be monitored in real time. For this real-time monitoring, it would be unnecessary to further operate the side surface optical fiber 350 and move it.

As illustrated in FIG. 19, the balloon 320 is inserted and expanded around the tissue to be treated in the trachea, and the optical fiber 340 in the balloon 320 emits laser to uniformly deliver laser to the tissue to be treated through the balloon 320, thereby the catheter-based laser treatment device 300 according to the preferred embodiment of the present invention as mentioned above allows photocoagulation for the target tissue.

In this case, the pressure controlling part 330 allows the drug on the surface of balloon 320 to be delivered to the target tissue by allowing the laser system 345 to emit laser to the tissue through the optical fiber 340 or vibrating the balloon 320 with a time difference, simultaneously.

Additionally, the catheter-based treatment device 300 according to the preferred embodiment of the present invention may perform monitoring by the side type optical fiber 350 right after or simultaneously with laser emission as illustrated in FIG. 20, which allows an operator to conduct photocoagulation more precisely and safely.

Meanwhile, it was explained as an example that the catheter-based treatment device 300 according to preferred embodiments of the present invention is used in the treatment of trachea. However, it is of course that the catheter-based treatment device 300 of the present invention may be used for the treatment of all tubular human tissues other than trachea.

4. Electromagnetic Energy Application Device for Tubular Tissue Stricture

The fourth aspect of the present invention relates to anelectromagnetic energy application device for tubular tissue stricture, which includes a catheter; a balloon catheter having an inner space interconnected with the catheter, connected to an end of the catheter enabling expansion and contraction; a pressure controlling part inserting or discharging operation fluid to introduce the operation fluid into the balloon catheter or discharge the operation fluid from the balloon catheter through the catheter; an optical fiber inserted into the balloon catheter penetrating through the catheter; a laser system transmitting laser through the optical fiber; and a location moving part withdrawing the balloon catheter.

According to the present invention, the front end of the balloon catheter is formed in a sharp funnel shape, or the front and rear ends are symmetrically formed in a sharp funnel shape.

The pressure controlling part of the present invention inserts or discharges the operation fluid at a pressure of 1 to 15 psi.

The pressure controlling part of the present invention vibrates the balloon catheter at a frequency of 1 to 100 Hz while maintaining a constant pressure.

The pressure controlling part generates a vibration wave, and the vibration wave is delivered to the balloon catheter through the operation fluid.

The pressure controlling part controls the insertion or discharge speed of the operation fluid so that the 1 and contraction speed of the balloon catheter is 10 to 1000 μm/sec.

The pressure controlling part vibrates the balloon catheter, simultaneously when the laser system emits laser to the tissue through the optical fiber.

Hereinafter, preferred embodiments of the electromagnetic energy application device for tubular tissue stricture according to the present invention will be explained in detail with reference to the drawings attached.

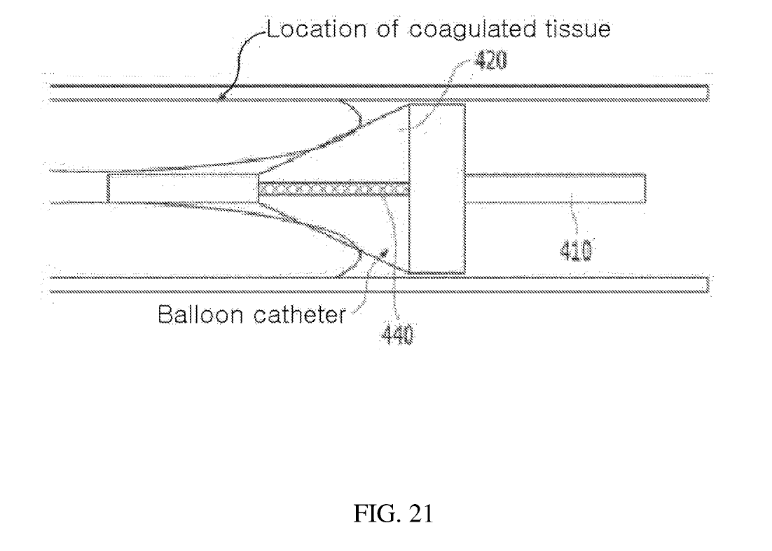

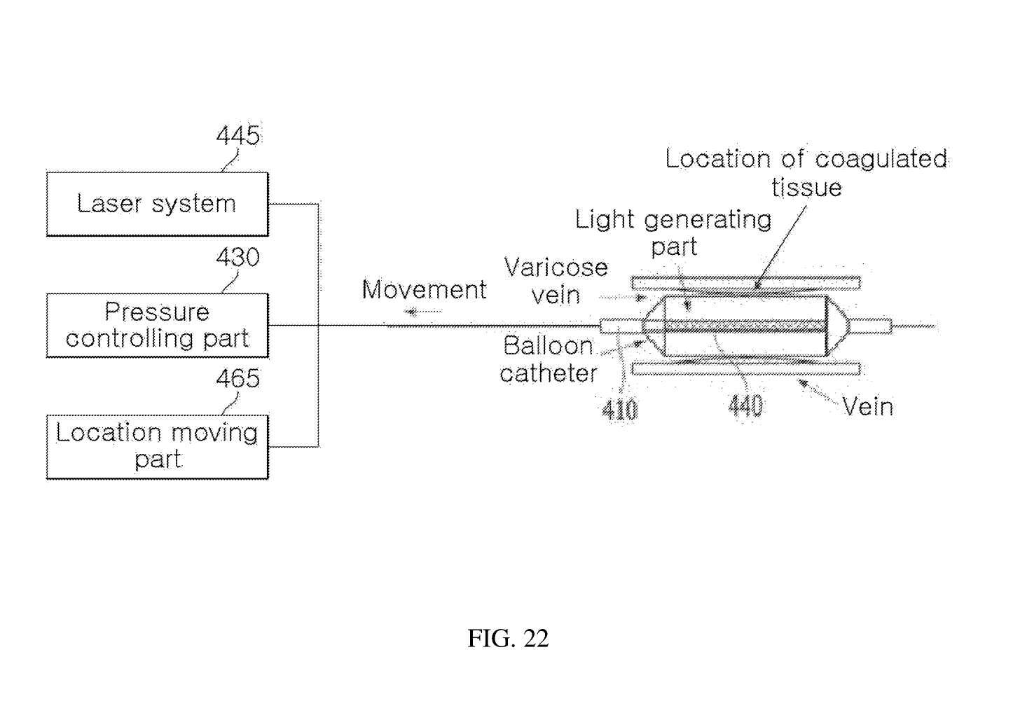

FIG. 21 is an exemplary view illustrating a state of proceeding with vascular stricture through a balloon catheter according to the present invention, and FIG. 22 is an exemplary view illustrating a process of proceeding with an optical treatment by inserting an optical fiber into the balloon catheter according to the present invention.

Hereinafter, the electromagnetic energy application device for tubular tissue stricture according to preferred embodiments of the present invention will be explained with reference to FIG. 21 and FIG. 22.

The electromagnetic energy application device for tubular tissue stricture according to the preferred embodiment of the present invention includes a catheter 310, a catheter balloon 420, a pressure controlling part 430, an optical fiber 440, a laser system 445, and a location moving part 450.

The catheter 410 is formed in a tubular shape and inserted into the body, and the optical fiber 440 is inserted through the inner penetrating path.

The balloon catheter 420 has an inner space interconnected with the catheter 410, and connected to an end of the catheter 410 to be form as expandable or contractible balloon.

The balloon catheter 420 is formed of a material from which laser ray emitted through the optical fiber 440 is penetrated into the tissue to be treated.

Additionally, the balloon catheter 420 is formed in geometrically various shapes, for example, the front end of the balloon catheter is formed in a sharp funnel shape, or the front and rear ends are symmetrically formed in a sharp funnel shape.

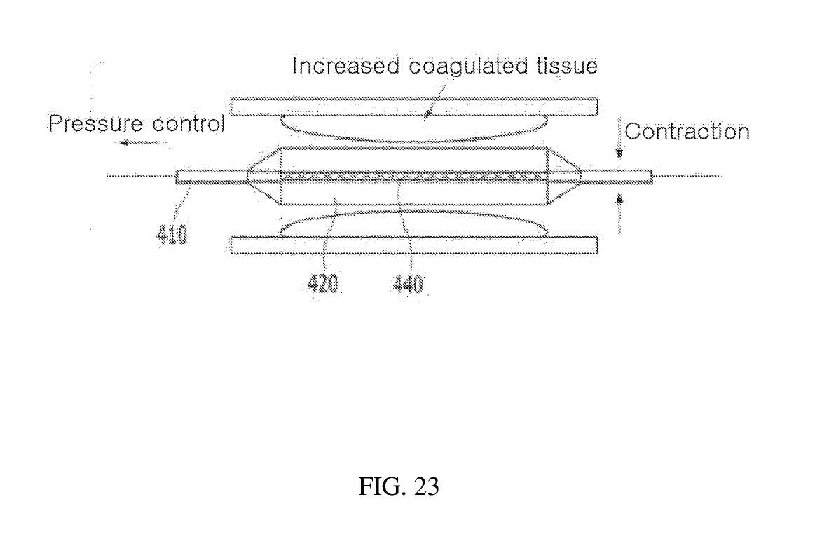

FIG. 23 is an exemplary view illustrating a state of continuously delivering uniform heat to a vessel wall by adjusting a pressure inside the balloon catheter as the blood vessel is adsorbed according to the present invention.

As illustrated in FIG. 23, the pressure controlling part 430 of the electromagnetic energy application device for tubular tissue stricture according to the present invention inserts or discharges the operation fluid to expand or contract the balloon catheter 420 through the catheter 410 to introduce the operation fluid to the balloon catheter 420 or discharge the operation fluid from the balloon catheter 420.

In this case, the operation fluid, for example, consists of fluid which is harmless to human body if it is inserted into trachea like air or saline solution. Additionally, the pressure controlling part 430 and catheter 410 are directly connected, or interconnected through an additional conduit so that the operation fluid could be flowed through the conduit.

The pressure controlling part 430 may be implemented with means such as a pump inserting or discharging the operation fluid. Preferably, the pressure controlling part may be implemented with an electronic pump capable of precisely controlling the amount of insertion or discharge of the fluid according to a predetermined speed.

Specifically, the pressure controlling part 430 controls the speed of insertion or discharge of the operation fluid so that the speed of expansion and contraction of the balloon catheter 420 could be 10 to 1000 μm/sec.

Additionally, the pressure controlling part 430 is capable of expanding or contracting the balloon 420 by inserting or discharging the operation fluid with a pressure of 1 to 15 psi.

The pressure controlling part 430 allows the balloon 420 to be expanded or contracted with various speeds and pressures. Additionally, the balloon 420 gives or releases the pressure to the tissue coagulated by the laser ray so that the corresponding tissue could be expanded or permanently modified.

At this time, when the speed of expansion and contraction of the balloon 420 is less than 10 μm/sec, the expansion and contraction speed are very slow, and thus it would be difficult to induce modification of tissue within a given time. When the speed of expansion and contraction exceeds 1000 μm/sec, the speed is excessively fast, and thus it would not be easy to control the pressure of the balloon 420 and the tissue could be damaged by a sudden expansion pressure of the balloon catheter 420.

Also, when the pressure expanding and contracting the balloon 420 is less than 1 psi, the pressure is so low, and thus it would be difficult to induce modification. It is possible to sufficiently modify the tissue in the trachea under the pressure below 15 psi, so the pressure exceeding 15 psi is unnecessary. The pressure exceeding 15 psi may excessively press the tissue so the tissue could be damaged.

Furthermore, the pressure controlling part 430 is configured to vibrate the balloon 420 at a frequency of 1 to 100 Hz while maintaining a constant pressure. The pressure controlling part 430 causes periodical expansion and contraction of coagulated tissue through the balloon 420 so that the trachea may permanently and easily control the size modified and modification rate.

For this, the pressure controlling part 430 may include a means for generating a vibration wave (not illustrated) generating a vibration wave, and the vibration wave generated by this is delivered to the balloon 420 through the operation fluid.

The pressure controlling part 430 is capable of repetitively inserting and discharging a small amount of operation fluid to the balloon 420 according to a fixed time interval in order to vibrate the balloon 420 with a fixed interval.

The optical fiber 440 is passed through the catheter 410. Additionally, one end thereof is inserted into the inside of the balloon 420, and the laser system 445 transmitting the laser through the optical fiber 440 is disposed at another end of the optical fiber 440.

The optical fiber 440 is formed of the optically diffusing fiber. Additionally, the probe or glass cap for diffusing or condensing laser ray with a proper form according to the necessity may be included in the one end of the optical fiber 440.

The laser system 445 is connected to the optical fiber 440 to supply laser ray, and the laser system 440 controls the wavelength of laser ray, emission strength and emission interval according to the properties of tissue to be treated.

The pulsed laser and continuous wave laser (cw laser) may be used as laser supplied to the optical fiber 440 by the laser system 445. As the wavelength of laser, a visible ray wavelength, a near infrared ray wavelength, a medium infrared wavelength, far infrared ray wavelength, etc. may be applied.

In this case, the laser system 445 may include a laser diode capable of modulating an output signal in order to control emission strength of laser ray through which the degree of penetration of laser ray into the tissue to be treated and temperature may be precisely controlled.

The location moving part 465 includes a step motor, etc., which is not illustrated, to move a location of the balloon catheter 420 in the back direction. Additionally, after operation, the location moving part withdraws the balloon catheter 420 within the blood vessel.

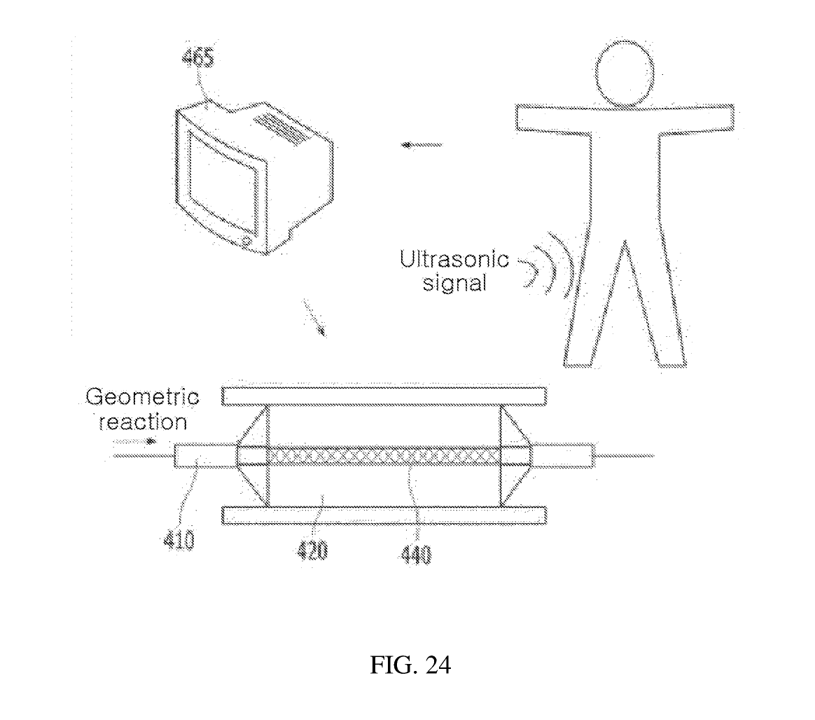

FIG. 24 is an exemplary view proceeding with a targeted treatment by expanding as long as a unique diameter of a blood vessel through monitoring according to the present invention.

As illustrated in FIG. 24, the electromagnetic energy application device for tubular tissue stricture according to the present invention is connected to the imaging system 450 using ultrasonic signal. The imaging system 450 transmits an ultrasonic signal to a part to be treated in human body through a ultrasonic signal generator, which is not illustrated, to receive the ultrasonic signal reflected, thereby obtaining an image of tissue of a part to which the balloon catheter 420 is inserted through the ultrasonic signal and outputting the image on a screen such as a monitor, etc.

Here, the imaging system 450 may be implemented with an image photographing device such as an optical coherence tomography (OCT) device, a photoacoustic tomography device, a polarization imaging device, etc.

Accordingly, the electromagnetic energy application device for tubular tissue stricture according to the present invention may perform monitoring by the imaging system 450 right after or simultaneously with laser emission which allows an operator to conduct photocoagulation more precisely and stably.

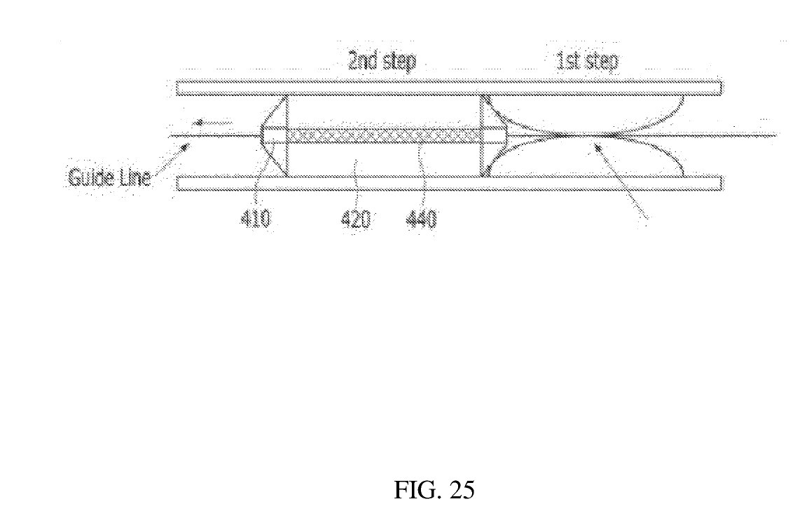

FIG. 25 is an exemplary view illustrating a treatment state of an entire blood vessel through a motion control after clarifying a treatment scope through a balloon catheter according to the present invention to proceed with a partial treatment.

As illustrated in FIG. 25, the electromagnetic energy application device for tubular tissue stricture according to the present invention obtains an image of tissue of a part to which the balloon catheter 420 is inserted through the ultrasonic signal, monitors the image through a screen like a monitor, etc., and controls the pressure controlling part 430 according to the contraction rate of blood vessel, thereby automating the contraction speed of the balloon catheter 420. Additionally, continuous heat is delivered to the blood vessel adsorbed through the contracting balloon catheter 420 may induce hemostasis. Also, the range of treatment is divided and the treatment is conducted per part, thereby increasing treatment rate and efficiency and reducing difference in treatment outcome resulting from skills.

Furthermore, the energy of laser supplied to the optical fiber 440 by the laser system 445 may be changed in consideration of various diameters, lengths, etc. of the blood vessel to control the amount of coagulation, thereby providing more convenient treatment technology.

Meanwhile, it was explained as an example that the electromagnetic energy application device for tubular tissue stricture according to the present invention was used for the treatment of trachea. However, it is of course that the electromagnetic energy application device for tubular tissue stricture according to the present invention may be used for the treatment of all tubular human tissues other than trachea.

The electromagnetic energy application device for tubular tissue stricture according to the present invention as described above uses various balloon catheters with geometric shapes to minimize hemorrhage by the blood vessel before or during the treatment using the expansion of balloon catheter, and induce stricture of blood vessel without contraction of the balloon catheter. Additionally, the electromagnetic energy application device for tubular tissue stricture may use a fixed form of balloon catheter to automatically induce deflation of catheter according to contraction of blood vessel during laser treatment.

### EXAMPLES

### 1. Introduction

Menorrhagia is an abnormality of having excessive bleeding from the uterus during a woman's menstrual cycle. On average, 30% of women experience heavy uterine bleeding at some time in their lifetime. Symptoms of menorrhagia may include heavy, prolonged or irregular periods of more than 80 ml blood loss. Women with menorrhagia can be treated medically with oral contraceptive pills, nonsteroidal anti-inflammatory drugs, and androgenic steroids, etc. However, these medications are often associated with various side effects as well as temporary relief. In order to seek a more permanent solution, surgical treatment alternatives have also been performed. A definitive treatment for menorrhagia and other gynecological diseases is a hysterectomy, removal of the uterus. Nevertheless, the procedure is quite radical and invasive, with possible accompanying hemorrhage, long recovery time, high infection rate, bowel obstruction, and even sudden hormonal change. Thus, patients with menorrhagia often pursue alternatives to hysterectomy.

As a less invasive treatment option, hysteroscopic endometrial ablation has instead been performed to treat menorrhagia by using a number of techniques such as thermal balloon, cryotherapy, bipolar radiofrequency, and microwave ablation. Endometrial hyperplasia is one of the major causes of heavy menstrual bleeding. Thus, throughout the ablative techniques, the endometrium, which is the innermost layer of the uterus, is surgically removed without damaging the myometrium, the outer layer of the endometrium in order to maintain fertility. In spite of minimally invasive procedures, these treatments are still technically difficult and may result in thermal injury to peripheral tissue, eventually leading to various complications and unwanted sterility. In addition, the procedures require a series of treatments of at least 10 minutes to complete endometrial ablation, postoperatively leaving severe pain. Evidently, surgeons still need a way to complete endometrial destruction without the need for general anesthesia, surgical intervention, and complications.

Due to their high degree of accuracy and safety, fiber-based lasers have proved to be useful tools to ablate the endometrium with varying degrees of success. A variety of wavelengths, including 805 nm for diode, 1064 nm for Nd:YAG, 1320 nm for Nd:YAG, and 2.12 μm for Ho:YAG, have been used for endometrial ablation. Through direct irradiation of optical energy, the endometrium can be coagulated due to light absorption and resultant heat accumulation, leading to coagulation necrosis. The diode laser presented overall tissue effects similar to those of Nd:YAG lasers, both experimentally and clinically in light of tissue necrosis. However, low absorption coefficients, particularly at near-IR (1064 and 1320 nm), resulted in deep optical penetration depth up to 5 mm in soft tissue (for water, optical penetration depth=1/absorption coefficient=1/0.1 cm-1=10 cm) and thus irreversible thermal damage into the deep tissue, entailing hemorrhage at the surface of the uterus. In addition, the lasers of 805, 1064, and 1320 nm were operated in the continuous wave (CW) mode, so irreversible thermal injury was aggravated by protracted irradiation time and long heat diffusion. Under the irrigation environment, the mid-IR wavelength (2.12 μm) was readily associated with transmission loss on account of saline absorption (absorption coefficient=70 cm-1), so the laser would require higher input power for efficient light delivery. Furthermore, end-firing fibers could hardly achieve uniform tissue coagulation due to their small numerical aperture (N.A.) to cover the endometrium surface and difficulty in maneuvering the fiber during laser ablation.

In an attempt to obtain homogeneous light distribution, an optical diffuser has been developed and evaluated for endometrial ablation. The diffuser was created by removing the cladding and adding a diffusing medium such as silicon and scattering particles on the core surface. However, the applied power level (≦25 W) was relatively lower than the requirement for surgical tissue removal. In addition, the procedure was cumbersome because it required long irradiation time as well as administration of photosensitizers into a body prior to the operation, in comparison with surgical treatments. To prevent the risk of melting a diffuser, particularly under high power application, a balloon catheter was also developed and used together with a near-IR laser for treatment. Since the laser heated the balloon material directly rather than the targeted tissue, indirect heating was induced to the endometrium layer, which needed the real-time monitoring of temperature inside the tissue with thermocouples for safety purpose. Additionally, a 1064 nm wavelength with deep optical penetration (˜5 mm) at lower power (20 W) contributed to long irradiation time (10 to 12 min), deep coagulation necrosis (up to 4 mm), and undesirable hemorrhage.

In the current study, an endoscopic optical diffuser was designed and developed for minimally invasive endometrial ablation with a visible wavelength. Due to high vasculature in the uterus, an effective wavelength of 532 nm was selected to target hemoglobin in blood vessels and glandular tissue in the endometrium and, in essence, to treat menorrhagia. A 1-mm core fiber was directly micro-machined to create scattering segments for light diffusion. The balloon catheter was incorporated with the diffuser in order to achieve fast and uniform heat distribution as well as to provide structure integrity during the treatment. Light propagation from the diffuser was optically simulated, and the designed diffusing device was evaluated in vitro and in vivo in terms of coagulation time and necrosis thickness. The prototype device was also validated with a cadaveric human uterus to see its clinical applicability.

### 2. Materials and Methods

### 2.1 Fiber Fabrication and Simulation

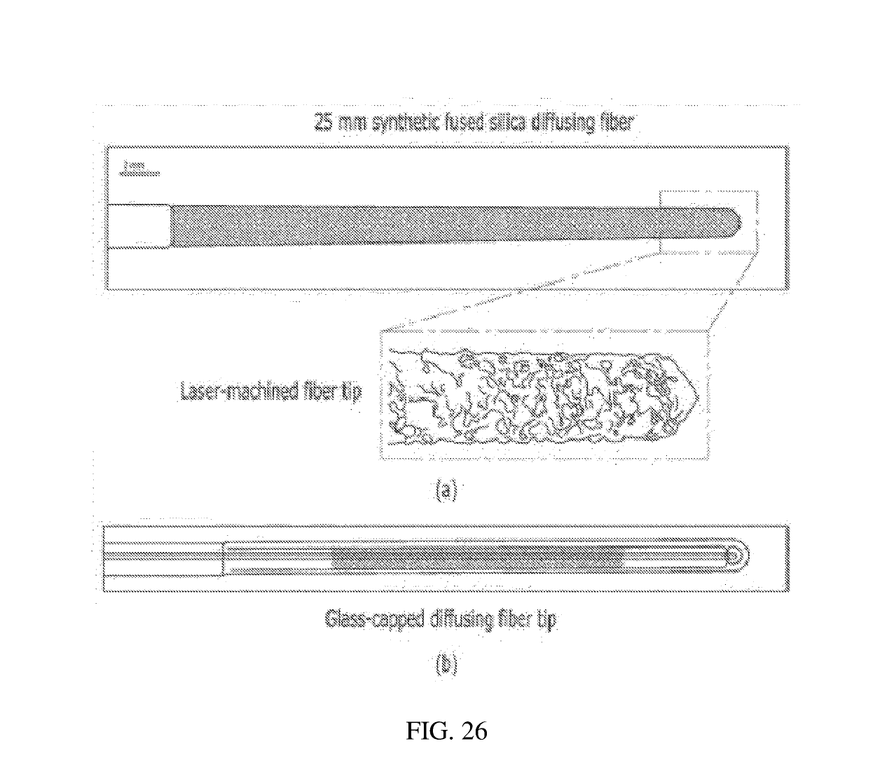

FIG. 26 presents images of a fabricated diffusing fiber tip for endometrial treatment. For simple and reliable machining purposes, a 1-mm core diameter, synthetic-fused silica was selected to transmit the visible laser light. Initially, the fiber cladding was removed mechanically, and the surface of the fiber core at the 25 mm distal end was circumferentially micro-machined by a 30 W CO2 laser in a predetermined zigzag pattern. A series of scattering segments were created on the fiber surface to diffuse the laser light radially in all directions. In order to achieve the constant size of the scattering segments, the fiber tip was tapered down to 0.5 mm in diameter at the distal end [i.e., minimum diameter for currently reliable manufacturability; FIG. 26(a)]. Then, the diffusing tip was covered with a 27-mm long glass cap to attain wide and uniform light diffusion as well as to protect the bare fiber tip during laser treatment.

In an attempt to predict photon distribution from the designed diffusing tip, optical simulation (Zemax) was conducted to demonstrate light intensity and its spatial distribution at various distances. Two fiber conditions were compared: a bare diffuser tip and a glass-capped diffuser tip. To model the light scattered from the fiber surface, a Lambertian diffuser model was used with one million of rays and a light source with uniform angular distribution. The diffusing tip was exclusively modeled with surface scattering (i.e., ˜50 μm size scattering segment). The applied wavelength was 532 nm with the input power of 120 W, and the entire fiber length was 1.5 m including a 25 mm diffusing part at the tip. A 40×50 mm planar detector was placed underneath the diffusing fiber at distances of 1, 5, and 10 mm to identify light propagation and the spatial distribution of the scattered photons in two-dimensional (2-D). The profiles of light intensity were also measured and quantitatively compared between the two fiber conditions.

### 2.2 In Vitro Experiments

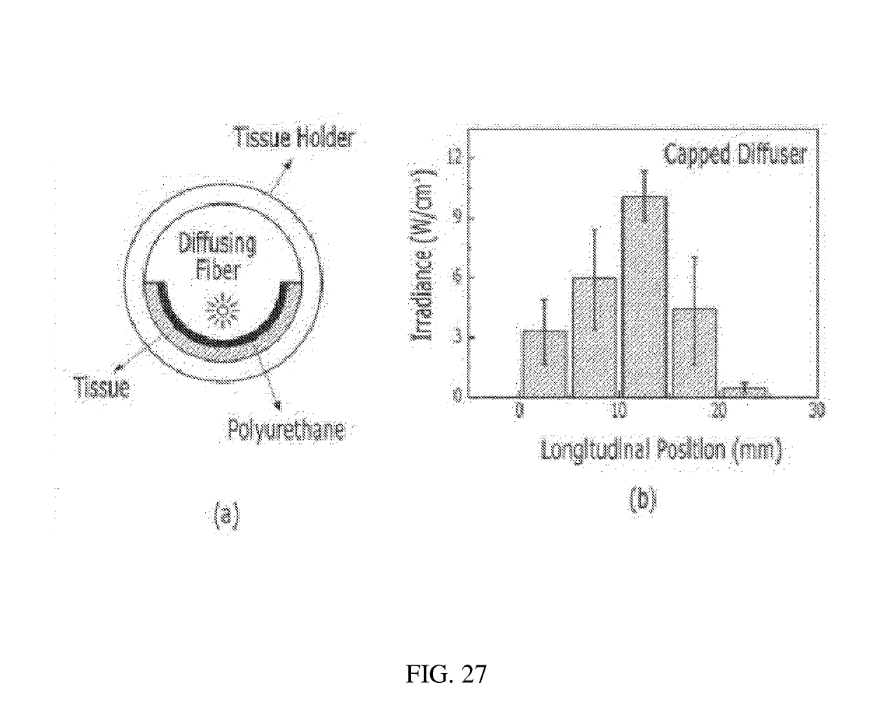

Bovine liver tissue was used as a tissue model for in vitro tests with the designed diffusing fibers, in that the chromophores, such as dead endothelial cells and blood vessels, would still be able to absorb the visible laser light (wavelength=532 nm) significantly. The liver specimens were acquired from a local slaughter house, and they were cut into 5×7 cm segments in size and 1 cm thick and stored at 4° C. prior to the experiments. FIG. 27(a) illustrates an experimental set-up of photocoagulation tests with the fibers. A circular tissue holder (7 cm in diameter and 1 cm in thickness) was prepared, and a 1 cm thick tissue sample was placed at the bottom of the holder along the curvature. The curved surface of the tissue sample partially reflected the transverse anatomic features of human uterus [FIG. 27(a)]. The diffusing fiber was located 1 cm above the tissue surface, so the incident light was uniformly irradiated on the tissue due to the curved geometry of the sample. A conventional clinical laser (532 nm) was employed for laser coagulation, and the input power was maintained at 120 W in order to entail tissue coagulation through the diffusing tip. The average irradiance on the tissue surface was calculated to be approximately 3.8 W/cm2. Three fiber conditions were tested: bare diffusing fiber, capped diffusing fiber, and capped diffusing fiber with a 1 mm thick layer of polyurethane (PUR). As PUR is a raw material for balloon catheters for endoscopic applications, so the last condition represented the final device design to incorporate the capped diffusing fiber into a balloon catheter. However, for the sake of experimental simplicity, a thin layer of PUR was placed on top of the target tissue instead of using a balloon catheter. During the tests, a PUR layer was superimposed upon the tissue sample as shown in FIG. 27(a) (left image). Prior to coagulation tests, the optical transmission of each fiber (bare diffusing, capped diffusing, and capped diffusing with a PUR layer) was measured with a photodetector to be 99%, 97% and 94.5%, respectively. FIG. 27(b) presents the intensity profile along the capped fiber tip that was measured every 5 mm. In addition, the transmission loss of PUR at 532 nm was measured with the detector to be less than 2.5%, which experimentally verified negligible light absorption at 532 nm by the PUR layer. The coagulation threshold was preliminarily determined for the three fiber conditions by varying irradiation times from 2 to 8 s (1 s increments; a sample per each condition). The onset of visible discoloration on tissue surface was considered the physical evidence of coagulation. A number of irradiation times (4, 7, 15, 30, 60, 90, 120, 150, and 180 s) were then evaluated for the three conditions to identify the temporal evolution of photocoagulation on the tissue surface. Due to difficulty of preparing fresh tissue specimens with a large uniform surface area, each condition was tested only with a single liver sample for evaluating surface coagulation. All the specimens were placed under saline environment during the laser irradiation, and the temperature of the liver tissue was maintained at approximately 20° C. Postexperimentally, the radial depth (i.e., into the tissue) and 2-D area of coagulation on the tissue surface were quantified and compared with an image processing software tool (Image J, National Institute of Health, MD, USA). The image in FIG. 27(a) showed a cross-sectional part of the photocoagulated tissue. In order to measure coagulation thickness, each irradiated specimen was cross-sectioned into three pieces, and the coagulation thickness of each piece was measured five times (n=15). The discolored zone represented coagulation necrosis and the red color was the preserved native tissue. A Student's t-test was used for statistical analysis and p<0.05 meant statistically significant.

### 2.3 In Vivo and Cadaver Experiments

Three mature female Saanen goats were used for in vivo retrograde laser coagulation studies. Animal procedures and care were conducted in accordance with a protocol approved by American Preclinical Service (APS) Institutional Animal Care and Use Committee (IACUC). Experiments, necropsy, and histology were performed at APS, and all surgical procedures were performed with the animals under general endotracheal anesthesia. A caprine uterus is typically bicornuate so two prominent uterine horns come together to form a short uterine body. Thus, six caprine uteri in total were tested for the current photocoagulation tests. Similar to a human uterus, the caprine uterine wall consists of two major tissue layers: endometrium and myometrium. The endometrium is a pseudostratified layer of epithelium on the luminal surface of the uterus, containing richly vascular loose connective tissue along with fibroblasts, macrophages, and mast cells, etc. The myometrium is two layers of smooth muscle separated by the stratum vasculare, which is a zone of large vessels (arteries, veins, and lymph vessels). For the current in vivo studies, the prototype device was evaluated to photocoagulate solely the endometrial layer, in that any thermal injury to the myometrium would adversely affect fertility. The prototype device consisted of a capped diffusing fiber, a PUR balloon catheter, a customized inflating tube (1 cm outer diameter and 8 cm long), and a customized inflating pump (variable pressure levels from 1 to 7 psi). The 4-cm long catheter device was inserted into the animal uterus, and the balloon catheter was distended with saline until it securely held the uterine wall (i.e., approximately 3 cm in balloon diameter at 5 psi). A 532 nm clinical laser was used with the input power of 120 W, and the irradiation time was approximately 30 s, based upon in vitro results (i.e., applied energy=3600 J and irradiance=3.2 W/cm2 assuming a 3-mm thick endometrium for photocoagulation). Postoperatively, all the animals were euthanized 2 h after the tests by euthasol injection. Immediately after euthanasia, each uterine horn was removed, fixed in 10% neutral buffered formalin, and embedded in paraffin for hematoxylin and eosin (H&E) staining. From histology images, the thickness of coagulation necrosis was measured with Image J (n=18) and evaluated quantitatively.

A human uterus was donated by a 59-year-old postmenopausal patient for research at APS after a radical hysterectomy. The cadaveric uterus was used to evaluate the feasibility of the prototype device in terms of light leaking and deployment of fiber and balloon during laser irradiation. The device was inserted through the cervix for minimally invasive uterine access, and a 5-cm long balloon catheter was distended at 4 psi by saline. The balloon catheter was approximately 1.8 cm in balloon diameter. The applied power of 120 W was irradiated on the uterine wall for approximately 20 s (i.e., applied energy=2400 J and irradiance=4.2 W/cm2) as the distance between the fiber and tissue was closer than the in vivo condition due to the rigidity of the cadaveric tissue. The degree of coagulation necrosis in the tissue was also examined postexperimentally with Image J (n=12). A digital camera (9.1M DSC-H50, Sony) was used to take images of pre-, intra-, and postoperation to show a sequence of photocoagulation. A Student's t-test was also used for statistical analysis and p<0.05 meant statistically significant.

### 3. Results

### 3.1 Optical Simulation

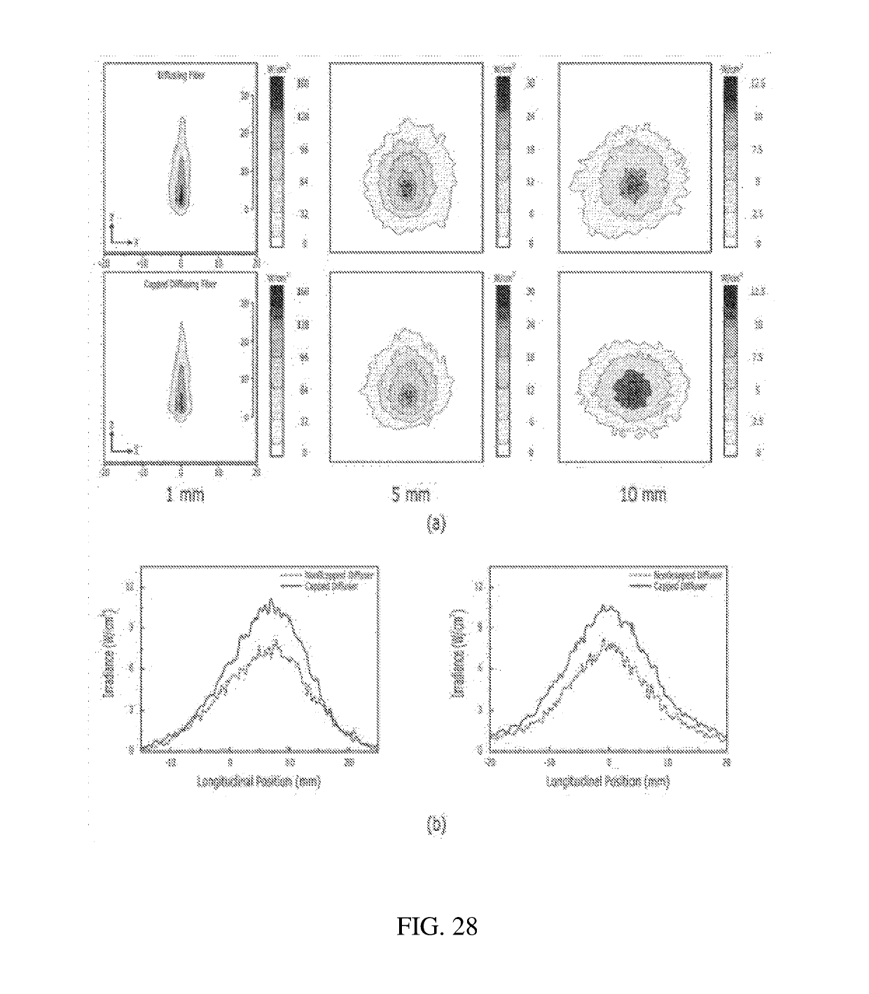

FIG. 28 shows the spatial distribution of photons from optical simulation comparing diffusing and capped diffusing fibers at various distances of 1, 5, and 10 mm. At 1 mm, both fibers created similar photon distributions with generation of high irradiance along the fiber due to their close proximity to a planar detector. However, as the distance from the detector increased up to 10 mm, the distribution became wider due to light diffusion from scattering segments on the fiber surface. The diffusing fiber presented elongated shape (along z axis) with relatively lower irradiance whereas the capped diffusing fiber created relatively circular distribution with higher irradiance, resulting from additional beam diffraction (along x axis) through the glass cap. At a distance of 10 mm, longitudinal and horizontal distributions of the incident photons were compared. Both directions demonstrated that the capped diffusing fiber yielded approximately 40% higher irradiance than the diffusing fiber (i.e., peak intensity=10.8 W/cm2 for the capped diffusing versus 7.7 W/cm2 for the diffusing fiber). Based upon the longitudinal position, the width of the irradiance distribution was comparable between the two cases. However, the capped diffusing fiber created around 30% wider horizontal distribution of the irradiance (i.e., Full-width-half-maximum=15.2 mm for the capped diffusing versus 11.5 mm for the diffusing fiber). According to the simulation results, an additional layer from the glass cap circumscribed the longitudinal photon distribution and contributed to uniformly distribute the incident photons along x-axis.

### 3.2 In Vitro Results

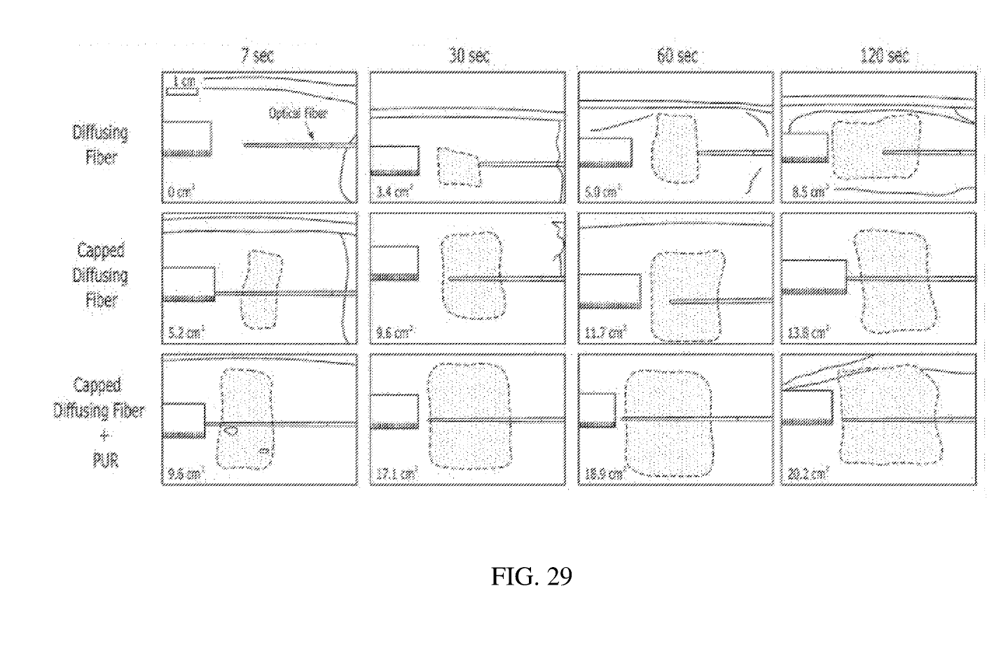

FIG. 29 demonstrates the progression of laser-induced coagulation on tissue as a function of irradiation time for three fiber conditions: diffusing, capped diffusing, and capped diffusing fiber with PUR. The total applied energy (i.e., 120-W input power times irradiation time) was 840, 3600, 7200, and 14,400 J at 7, 30, 60, and 120 s, respectively. Overall, the degree of photocoagulation on the tissue surface developed gradually with the irradiation time. Coagulation initially developed in a vertical direction (i.e., perpendicular to the fiber axis) and later expanded horizontally (i.e., along the fiber axis) with the irradiation time. The shape of the coagulated area was almost rectangular for the three conditions, and at 120 s the area length (i.e., perpendicular to the fiber axis) and width (i.e., along the fiber axis) were measured to be 3.1 and 2.5 cm, respectively. In other words, the area length was equivalent to the length of the half arc length of the diffusing light 1 cm away from the fiber, and the area width became equivalent to the length of the diffusing fiber tip. Compared to the diffusing and capped diffusing fibers, the last condition (capped diffusing fiber with PUR) overtly yielded more rapid and wider tissue coagulation (i.e., 9.6 cm2 for the last condition versus 0 cm2 for diffusing fiber and 5.2 cm2 for capped diffusing fiber at 7 s after irradiation). In fact, the coagulated area for the last condition became rapidly saturated after 30 s, compared to the two other conditions.

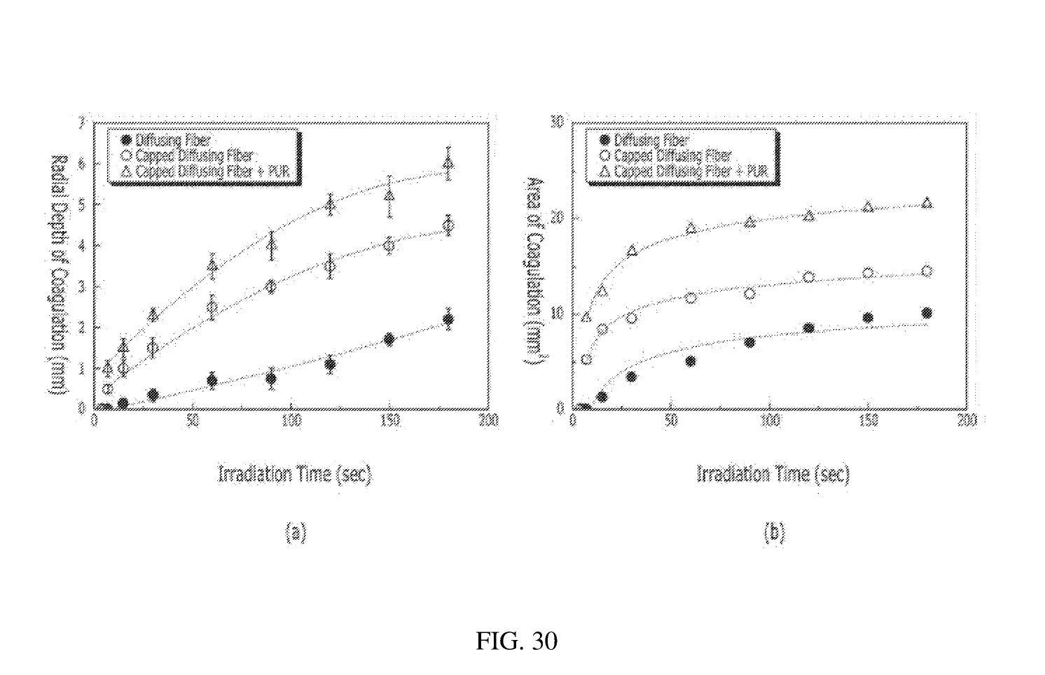

FIG. 30(a) shows the quantitative evaluation of coagulation depth in a radial direction (into the tissue) as a function of irradiation time. For a diffusing fiber, coagulation threshold time was around 7 s, whereas coagulation for two other conditions was initiated around 4 s after irradiation (coagulation thickness=100 to 200 μm). Similar to FIG. 29, a capped diffusing fiber with PUR increased the coagulation depth more rapidly than the other conditions. In the case of 1-min irradiation, the capped fiber with PUR created the coagulation necrosis of 3.5±0.3 mm, which was 5-fold and 1.5-fold thicker than the diffusing (0.7±0.2 mm) and capped diffusing fibers (2.5±0.3 mm), respectively [p<0.001; FIG. 30(a)].

FIG. 30(b) demonstrates the progression of coagulation area on the tissue surface that indicated lateral thermal expansion. The diffusing fiber presented that coagulation area increased almost linearly with the irradiation time as coagulation depth did. On the other hand, for both capped diffusing fiber and capped diffusing fiber with PUR, the coagulation area initially increased but became saturated approximately 1 min after irradiation, whereas the overall tendency of the coagulation depth almost linearly increased with time for both cases. At 1-min irradiation, the capped diffusing fiber with PUR condition yielded 3.8-fold and 1.6-fold larger coagulation areas than the diffusing and capped diffusing fibers, respectively (i.e., 18.9 cm2 for capped diffusing fiber with PUR versus 5.0 cm2 for diffusing and 11.7 cm2 for capped diffusing fibers).

### 3.3 Prototype and In Vivo Results

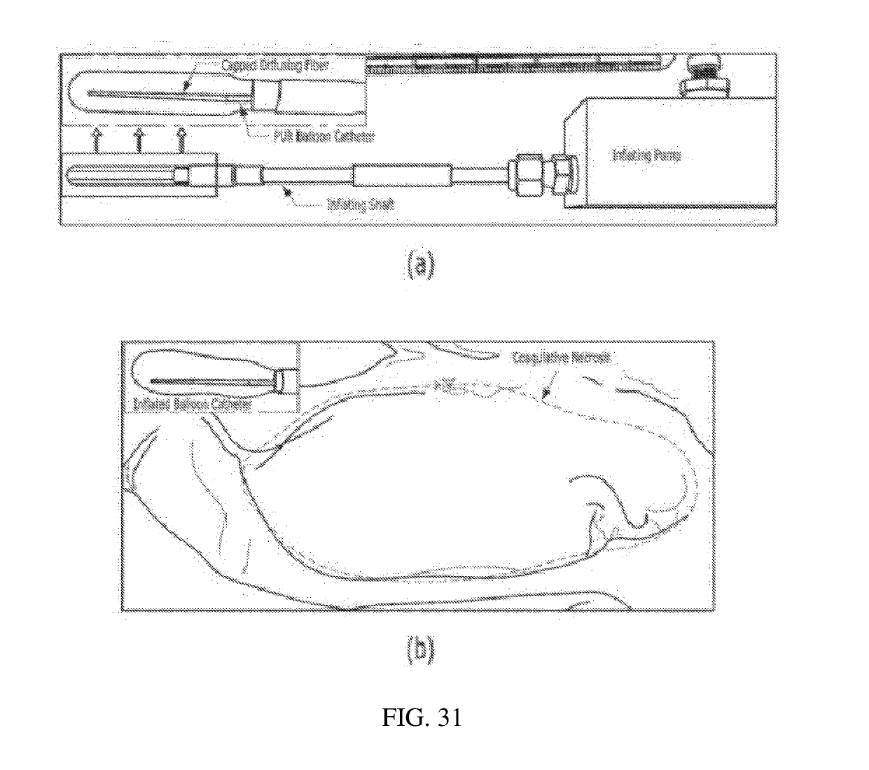

After in vitro validation of various diffusing fiber conditions, the design for the capped diffusing fiber was finalized, and the prototype optical device was made and incorporated with a balloon catheter for in vivo and cadaver studies as shown in FIG. 31(a). The capped diffusing fiber was placed in the center of an 8 cm long customized inflating tube, of which the proximal end was connected to a customized inflating pump (variable pressure levels from 1 to 7 psi) to regulate the input pressure for saline supply. The distal end of the fiber tip was insecurely positioned inside the balloon. A 4 cm long PUR-based balloon catheter was tightly attached to the distal end of the inflating tube, and the size of the balloon was adjustable, depending upon the geometry of uterus and pump pressure levels. In order to distend the balloon catheter, saline was pumped through the tube to fill out the balloon until the target uterus was securely fixed for laser surgery. Prior to in vivo tests, the prototype was validated to ensure the tight sealing of the catheter connection at the distal end of the tube.

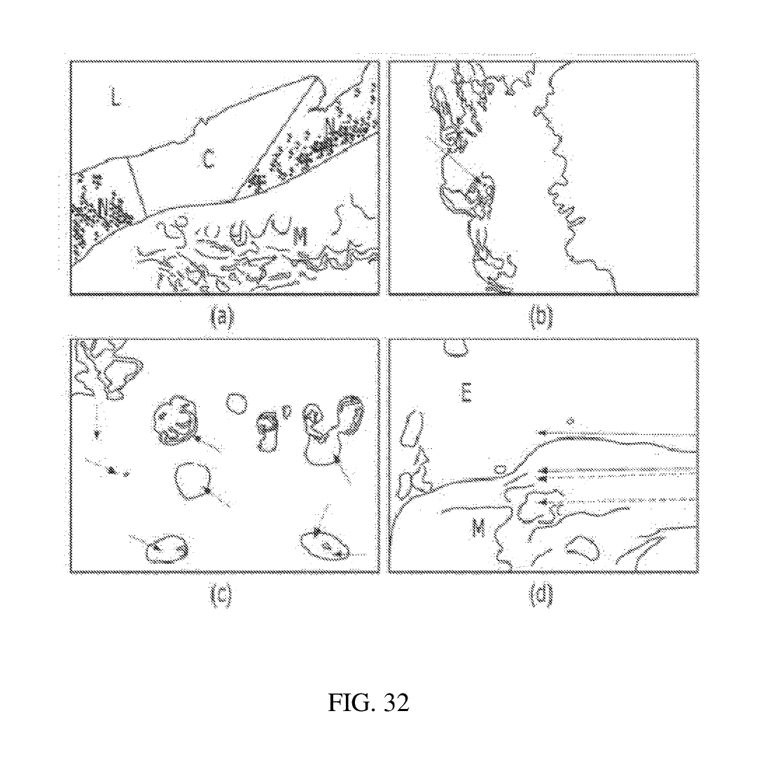

FIG. 31(b) shows the acute thermal response of a caprine uterine horn tissue 2 h after a 30 s coagulation with the prototype device (3600 J, 3.2 W/cm2). After 30 s irradiation, photocoagulation entailed the uniform coagulation necrosis on the treated tissue, which appeared as the shape of the distended (i.e., 3 cm wide and 4 cm long) balloon catheter [FIG. 31(b)]. The overall thickness of coagulation necrosis was measured to be 2.8±1.2 mm (n=18), which was slightly thinner than the estimated thickness (3 mm) of the distended uterine wall (p=0.53). No hemorrhage occurred in both endometrium and myometrium after the laser treatment. FIG. 32 presents H&E-stained histology images of the laser-treated uterine tissue. Treatment sites were readily identified by the presence of endometrial gland changes, edema, endometrial connective tissue changes, and vacuolation of smooth muscle cells in myometrium. The balloon-contacted tissue was merely coagulated without any thermal injury to adjacent tissue [FIG. 32(a)], in that endometrial glands, as well as the superficial epithelial lining on the luminal surface, were markedly obliterated along with sloughed epithelium. The coagulation process was also evidenced by protein coagulum created on the endometrium surface [FIG. 32(b)], smeared or atypical appearing epithelial cells [FIG. 32(c)], and amphophilic connective tissue surrounding vessels [FIG. 32(c)]. It was confirmed in FIG. 32(d) that the myometrial smooth muscle adjacent to the treatment site yielded minimal changes, such as cellular vacuolation and slight discoloration, representing no overt thermal damage or necrosis to the myometrium.

### 3.4 Cadaver Study

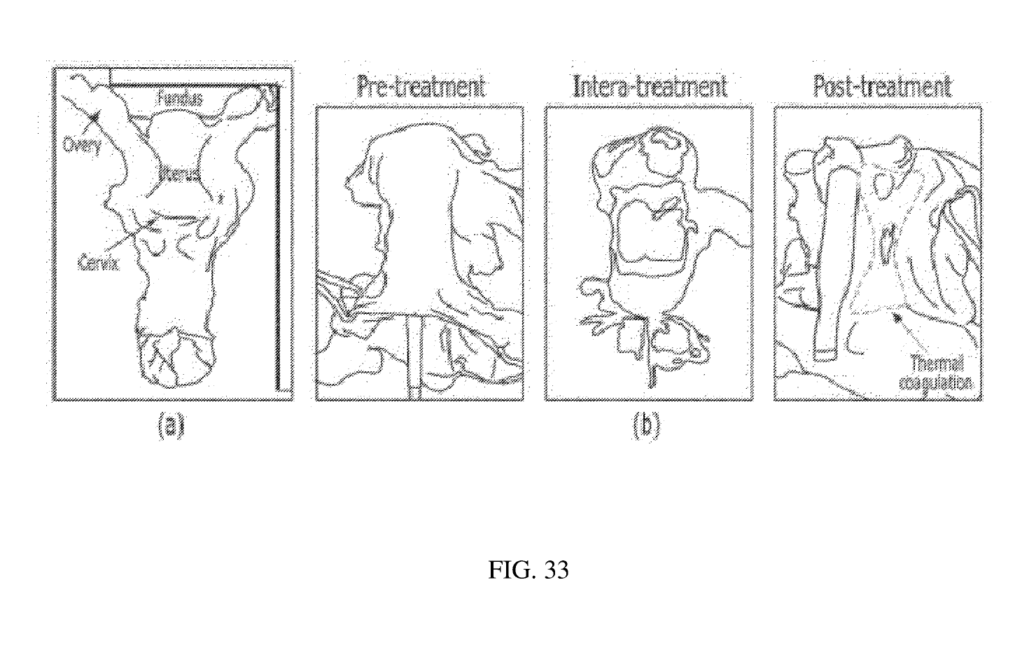

Followed by in vivo studies, a cadaveric human uterus was tested with the prototype device (2400 J, 4.2 W/cm2, FIG. 33). Unlike the in vivo caprine studies, a slightly longer balloon catheter (i.e., 5 cm long versus 4 cm long for in vivo) was used for the larger size of the human uterus [FIG. 33(a)]. However, due to the rigidity of the cadaveric tissue, the balloon was distended only up to 1.8 cm in diameter. Accordingly, the irradiation time was limited to approximately 20 s (i.e., 30% shorter than the irradiation time for in vivo study, 30 s) because of the closer irradiation distance between the fiber and endometrial luminal surface (i.e., 0.9 cm for cadaver study versus 1.5 cm for in vivo study) and thereby, its 30% higher irradiance. FIG. 33(b) shows a sequence of endometrial coagulation with the prototype balloon catheterassisted diffusing device. Prior to the treatment, the device securely held the uterus [FIG. 33(b)], and during the treatment, the scattered photons 532 nm were visualized through the tissue, indicating the ongoing intra-operation. According to posttreatment (far right image), the overall thickness of coagulation necrosis was found to be 2.6±0.6 mm (n=12). The coagulation thickness slightly thinner than the in vivo results indirectly evidenced that the adjusted dosimetry for the cadaver study was able to induce photocoagulation effects equivalent to those for the in vivo studies (i.e., 3.2 W/cm2 and 30 s irradiation).

### 4. Discussion

Spatial distribution of photons between diffusing and capped diffusing fibers were simulated and compared at various distances (FIG. 28). The current simulation models used a planar detector that displayed the gradient distribution of irradiance only in 2-D. However, the inherent anatomy of human uterus can be rather circular or doughnut shape. In turn, the intensity of the incident laser light can be constantly maintained along x-axis as the laser light is uniformly irradiated on the curved uterine wall at a constant distance with the aid of a concentric balloon-catheter. Unlike FIG. 28(b), the slope of the wings on the horizontal position would flatten out if one utilized the circular detector with the equivalent curvature that the diffuser had. Future investigations will be conducted to verify the physical distribution of the incident irradiance along the curvature of the uterine wall. In addition, the role of the glass cap layer will be studied to optimize the light distribution in light of layer thickness, curvature, and refractive index of the glass cap.

A capped diffusing fiber with PUR induced rapid and wide tissue coagulation as shown in FIGS. 29-31. Both the wide distribution of the incident photons from the glass cap and inhomogeneous illumination from the diffuser on the tissue surface could be responsible for lateral thermal expansion particularly with longer irradiation times, leading to the wide coagulation. It is also conceived that the enhanced coagulation was associated with thermal insulation of the PUR material. As the target tissue heated up upon light absorption, the PUR layer behaved as a thermal barrier to trap and accumulate the laser-induced heat inside the tissue. An insignificant amount of heat could barely diffuse through the PUR layer, in that thermal conductivity of PUR is almost 25-fold higher than that of water. In an attempt to ensure the efficacy and safety of the enhanced photocoagulation, numerical simulation on temperature development and distribution in tissue is underway to optimize the design dimension and material properties of PUR. For the sake of experimental validation, tissue temperature during laser irradiation with the optical diffuser will also be quantified with thermocouples embedded in tissue.

Based upon the assumption that an endometrial layer was 3 mm thick, the irradiation time for in vivo experiments was selected as 30 s to generate the coagulation thickness comparable to the endometrium thickness [FIG. 30(a)]. Although the typical thickness of the endometrial layer in human uterus is approximately 5 mm, the uterine wall can become thinned out owing to the expansion of the uterus during laser surgery. Thus, to ensure that the laser light solely ablates the endometrium without any thermal damage to the subjacent myometrium, the wall thickness was conservatively assumed to be 3 mm, roughly corresponding to the irradiation time of 30 s as shown in FIG. 30(a). In fact, the in vivo results evidenced that the measured coagulation thickness was comparable to 3 mm (FIG. 32). Thus, the conservative assumption on the distended uterine wall must have been valid and safe enough to protect the layer underneath the endometrium. Chronic response of uterine tissue will be further evaluated to investigate the histopathologic evolution of the treated uterine tissue as well as its potential healing patterns.

Considering a typical uterus volume, the estimated average irradiance on the entire uterus surface area (i.e., 88 cm2 assuming a uterine cavity as a frustum of right circular cone) would be 1.3 W/cm2 under 120-W application. The estimated value is approximately 70% lower than the irradiance (4.2 W/cm2) used for the cadaver study. Thus, one may need a longer irradiation time in order to achieve the comparable coagulation thickness. Moreover, since the uterus is a closed volume, diffuse reflection from the uterine wall could take place to uniformly distribute the diffusely scattered light, subsequently expanding thermal diffusion. Accordingly, one may have to take into account the effect of diffuse scattering on the uterine wall in an attempt to determine the appropriate irradiation time for clinical tests. It was also noted that a certain part of the treated tissue was superficially carbonized [FIG. 33(c)]. Since the diffusing fiber was attached only to the distal end of the inflating tube, insecure location of the diffusing fiber tip could be responsible for the unwanted carbonization as the fiber tip freely moved around in the distended balloon catheter even during/after irradiation. Furthermore, under the current study, 120-W input power was applied to yield enough irradiance through the diffusing tip and to entail tissue coagulation. Although high laser power has clinically been used, undesirable fiber failure would be detrimental to peripheral tissues, organs, and eventually patients. Thus, precautions such as fiber shield and optical feedback system should be considered for the new design to ensure safety during laser treatment.

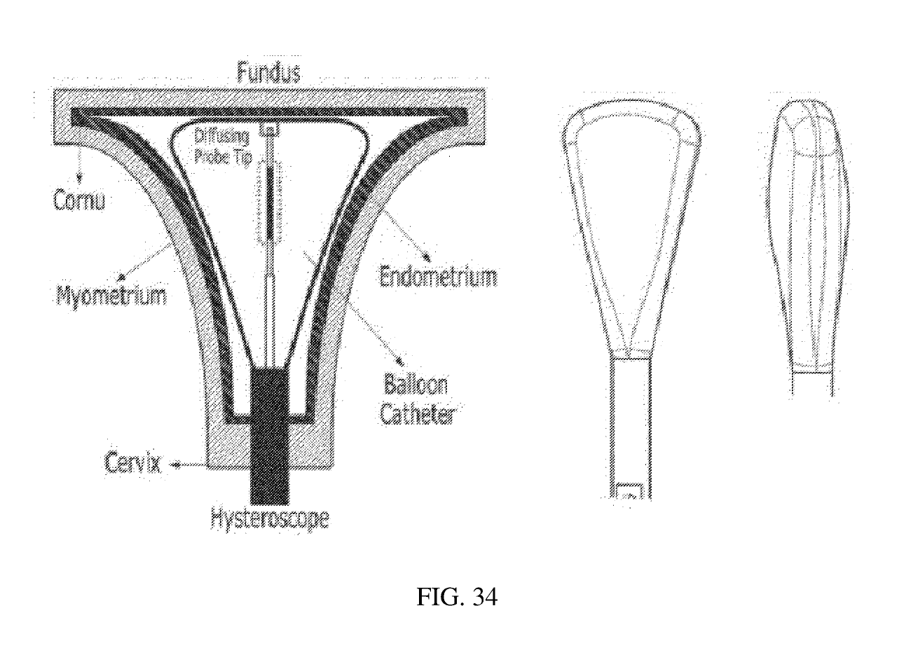

Since a cylindrical shape of the balloon catheter was used to make the prototype, the entire surface of the endometrium hardly achieved the uniform coagulation. Moreover, the anatomy of human uterus seems triangular. In an attempt to resolve the current challenges such as free movement of fiber tip and diverse uterus geometry, the new design for the optical device has been suggested and under investigation (FIG. 34). Firstly, a small holder is placed in the ceiling inside the balloon to fix the position of the diffusing fiber tip, so the optical diffuser can stay in the center of the catheter even during device deployment and laser irradiation. Secondly, the shape of the balloon catheter is redesigned to be triangular, so the entire balloon can increase the light coverage of the endometrium surface. Finally, along with the new geometry of the balloon, the light distribution should change by providing the gradient of light intensity from different shapes of scattering segments on the fiber surface. In other words, more light can be concentrated on the upper part of the balloon, so the entire irradiance can be uniform over the inner surface of the balloon catheter. Further, preclinical and clinical evaluations will validate the performance of the new optical device for endometrial treatment.

The current study demonstrated the laser irradiation time on the order of seconds to treat endometrial cell layers (FIGS. 29, 31 and 33). Although the results may satisfy the clinical unmet need for quick treatment raised by gynecologists and patients, the real clinical situations would take much longer to complete treatment due to various sizes of distended human uterus and resultantly lower irradiance on the uterine wall. From the safety perspective, the input power lower than the current power used would be more desirable for clinical treatments to prevent any adverse events caused by fiber failure. Accordingly, the new design of the diffuser tip should take into consideration ways to optimize laser and fiber parameters such as low input power, high transmission, and uniformity of light distribution. Furthermore, in an attempt to enhance both clinical efficacy and safety, other minimally invasive techniques such as PDT have also been studied for treating AUB. Particularly, as PDT successfully induced endometrial destruction in a rat model, the newly proposed optical device can be compared and evaluated with PDT in terms of treatment performance and safety. Additional investigations will thereby be planned to verify the feasibility of incorporating two minimally invasive treatments into one potential therapeutic tool to amplify clinical outcomes.

### 5. Conclusion

The feasibility of the newly designed diffusing optical device was demonstrated for endometrial treatment. Due to the wide distribution of photons with high irradiance, the new optical diffuser was incorporated into a balloon catheter facilitated photocoagulation globally, compared to other minimally invasive techniques. The optical response of uterine tissue to 532 nm irradiation confined tissue coagulation to endometrial cell layers without any thermal injury to myometrium, unlike Nd:YAG lasers that cause deep coagulation necrosis. The uniform and rapid development of coagulation (2 to 3 mm thick) evidenced that the balloon catheter-based optical diffuser can be exploited to treat heavy menstrual bleeding as a simple and safe therapeutic device. Further development of the proposed design may provide a more efficient and safer tool for gynecologists to treat menorrhagia as well as other uterine diseases in a minimally invasive way and eventually to minimize postoperative complications.

The present invention uses the optically diffusing fiber capable of emitting light in a plurality of directions to apply to photothermal treatment or photodynamic therapy through an insertion into an inner tissue of human body. Additionally, the optically diffusing fiber may be used for treating thyroid cancers, breast cancers, prostate cancers, kidney cancers, bladder cancers, brain tumor, inner uterine wall, localized liver cancers, skin cancers, cancer tissue, coagulation of inner tissue, removal of fat, etc.

Additionally, according to hybrid optical medical equipment for both diagnosis and treatment of tubular human tissue according to the present invention, acquisition of OCT images for tubular body tissue such as trachea, blood vessel and ureter, and induction of photothermal treatment of body tissue by laser may be integrally performed through a single probe, thereby increasing efficiency of lesion diagnosis of tubular body tissue and induction of treatment. Also, the OCT image for the body tissue may be monitored in real time before and after performing the induction of photothermal treatment of body tissue, thereby efficiently performing diagnosis for lesion tissue and induction of treatment while minimizing damage on body tissue. Especially, diagnosis for every respiratory disease such as asthma and an induction of treatment may be promoted.

Also, the catheter-based laser treatment device according to the present invention has effects of preventing tracheal stricture from being recurred after surgery, and minimizing complications such as inflammation, injection, etc. which may be occurred during recovery.

According to the present invention, the use of various balloon catheters with geometric shapes may minimize hemorrhage by a blood vessel before or during treatment by using expansion of the balloon catheter, and induce vascular stricture without contraction of the balloon catheter.

The present disclosure is described with reference to the above embodiments. It should be appreciated by those skilled in the art that various changes and modifications may be made to the embodiments without departing from the scope of the present disclosure. Thus, the described embodiments set forth above are intended solely for explanatory purposes, not for limiting the present disclosure. The scope of the present disclosure is defined by the claims below. It should be appreciated that the present disclosure is not limited to the above embodiments, and all changes and/or equivalents thereto also belong to the scope of the present disclosure.

What is claimed is:

1. An optically diffusing fiber, comprising:
a fabrication length of a tissue treatment section required for laser treatment;
a tapering angle and an end diameter within the fabrication length capable of uniformly delivering optical energy;
a fabrication angle and fabrication part interval capable of varying optical energy distribution delivered; and
a height of an optically diffusing surface fabricated to vary a diffusion range of optical energy.

2. An optical fiber probe for treating a tubular tissue disease or a solid cancer comprising the optically diffusing fiber according to claim 1.

3. A method for manufacturing an optically diffusing fiber probe, comprising the following steps:
(a) inputting fabrication values including an optically diffusing range according to a disease part to be treated, energy distribution, optical fiber fabrication length, tapering angle, end diameter, fabrication angle, fabrication part interval, and height of an optically diffusing surface for manufacturing a suitable optical fiber for treatment length, etc.;
(b) outputting a fabrication control signal through a fabrication controlling part;
(c) fabricating a side surface and a front end of an optical fiber by moving the optical fiber in the rotational direction and front and back direction according to the fabrication control signal;
(d) delivering optical energy to an optical fiber;
(e) measuring optical energy delivered to the side surface and front end of an optical fiber through a side surface optical sensor and a front optical sensor; and
(f) determining whether to go through additional fabrication and polishing by comparing the measured strength with the pre-stored energy distribution of the optical fiber.

4. The method of claim 3, wherein the step (f) further comprises the step of conducting a feedback for precise fabrication when determined to go through an additional fabrication, and fabrication delivery speed, rotational speed, and fabrication energy are minutely controlled during the precise fabrication.

5. The method of claim 3, wherein the step (a) further comprises the step (a-1) of controlling the fabrication length L of the optical fiber in consideration of the tissue treatment section required for laser treatment, and the step (a-1) determines an initial fabrication location of the optical fiber with an overall fabrication length in consideration of a translational stage.

6. The method of claim 5, wherein the step (a) further comprises the step (a-2) of determining the tapering angle α and end diameter d of the optical fiber so that light of the optical energy is uniformly delivered through the optical fiber, and the step (a-2) determines the tapering angle α and end diameter d of the optical fiber by simultaneously or independently controlling the translational speed, rotational speed, power of fabrication energy source (0.1 W to 50 W), and area of energy source of the optical fiber.

7. The method of claim 6, wherein the step (a) further comprises the step (a-3) of determining a fabrication angle β and a fabrication part interval w to vary the optical energy distribution delivered through the optical fiber, and the step (a-3) determines the fabrication angle β and fabrication part interval w by simultaneously or independently controlling the translational speed and rotational speed of the optical fiber.

8. The method of claim 7, wherein the step (a) further comprises the step (a-4) of determining the height p of the optically diffusing surface to vary the diffusion range of optical energy light through the optical fiber, and the step (a-4) determines the height p of the optically diffusing surface by controlling the rotational speed of the optical fiber, power of fabrication energy source (0.1 W to 50 W) and area of energy source.

9. Hybrid optical medical equipment for both diagnosis and treatment of a tubular human tissue comprising:
a probe moving by being inserted in a tubular human tissue;
a human activating optical fiber module protruding to the front end of the probe by passing an inner passage of the probe, the human activating optical fiber module performing any one selected from obtaining an optical coherence tomography (OCT) image of a tubular human tissue through infrared light emission of a predetermined wavelength area and inducing tubular human tissue photothermal treatment through laser emission;
a controller connected to the human activating optical fiber module, performing the operation control of the human activating optical fiber module for obtaining an OCT image of human tissue and for inducing human tissue photothermal treatment; and
an OCT image output device connected to the controller, outputting an OCT image obtained from the human activating optical fiber module,
wherein OCT image monitoring on the tubular human tissue and laser stimulation thereon are performed integrally.

10. The equipment of claim 9, wherein the human activating optical fiber module performing tubular human tissue photothermal treatment inducement through the laser emission comprises an optically diffusing fiber.

11. The equipment of claim 9, wherein the human activating optical fiber module comprises:
an optical fiber for diagnosis emitting near infrared ray in a wavelength range of 800 to 1550 nm to a tubular human tissue and inducing obtainment of an OCT image for a predetermined part of a tubular human tissue through location adjustment by near infrared ray emission by translational movement and rotational movement; and
an optical fiber for treatment emitting laser of a predetermined wavelength to a lesion part of a tubular human tissue in a predetermined pattern, and stimulating the lesion part through location adjustment by laser emission by translational movement and rotational movement,
wherein the optical fiber for treatment is at least one selected from one optically diffusing fiber emitting near infrared ray from an entire part of an outer circumference, and at least one side type optical fiber emitting near infrared ray only to a predetermined area limited in the lateral direction.

12. The equipment of claim 11, wherein the human activating optical fiber module comprises an optical fiber integrated coating body formed of a penetrating path for movably receiving the optical fiber for diagnosis and optical fiber for treatment independently, so that the optical fiber integrated coating body passes the inner passage of the probe.

13. The equipment of claim 11, wherein the optically diffusing fiber is inserted into a balloon-shaped catheter passing through the inner passage of the probe and protruding to the front end of the probe, the balloon-shaped catheter having a balloon-shaped expansion tube arranged expandably at the end.

14. The equipment of claim 9, wherein the human activating optical fiber module comprises a single mode optical fiber which emits at least one selected from near infrared ray in a wavelength range of 800 to 1550 nm and laser of a predetermined wavelength to a tubular human tissue, controls the emission location by translational movement and rotational movement, and integrally performs inducement of obtainment of an OCT image for a predetermined part of the tubular human tissue and stimulation of a lesion part of the tubular human tissue.

15. The equipment of claim 9, further comprising:
a camera having a photographing lens forming exposure towards the front end of the probe; and
an optical source module for photography emitting visible rays through optical source bodies forming exposure towards the front end of the probe,
thereby performing macroscopic monitoring of a tubular human tissue through a tubular human tissue image photographed by the camera and microscopic monitoring of the tubular human tissue through the OCT image, simultaneously.

16. The equipment of claim 9, wherein the controller comprises:
a controller for tissue diagnosis performing the operation control of the human activating optical fiber module for obtaining an OCT image of a human tissue; and
a controller for laser treatment performing the operation control of the human activating optical fiber module for inducing photothermal treatment of the human tissue, and allowing Q-switched laser or pulse type laser in a wavelength of 300 to 3000 nm to be emitted on a tubular human tissue having hemoglobin over a predetermined level.

17. The equipment of claim 9, wherein the controller comprises:
a controller for tissue diagnosis for performing the operation control of the human activating optical fiber module for obtaining an OCT image of a human tissue; and
a controller for laser treatment performing the operation control of the human activating optical fiber module for inducing photothermal treatment of the human tissue, and allowing Q-switched frequency-doubled Nd:YAG 532 nm laser to be emitted on a tubular human tissue having blood vessel over a predetermined level.

18. The equipment of claim 9, wherein the controller comprises:
a controller for tissue diagnosis for performing the operation control of the human activating optical fiber module for obtaining an OCT image of a human tissue; and
a controller for laser treatment performing the operation control of the human activating optical fiber module for inducing photothermal treatment of the human tissue, and allowing laser in a wavelength of 800 nm to be emitted on a tubular human tissue injected with a bio-dye material, indocyanine green.

19. A catheter-based laser treatment device, comprising:
a catheter;
a balloon having an inner space interconnected with the catheter, connected to an end of the catheter enabling expansion and contraction;
a pressure controlling part inserting or discharging operation fluid to introduce the operation fluid into the balloon or discharge the operation fluid from the balloon through the catheter;
an optical fiber inserted into the balloon penetrating through the catheter;
a laser system transmitting laser through the optical fiber;
a side type optical fiber inserted into the balloon penetrating through the catheter; and
an imaging system transmitting and receiving light through the side type optical fiber to obtain an image of a tissue with the balloon inserted.

20. The device of claim 19, wherein the optical fiber inserted into the balloon penetrating through the catheter is an optically diffusing fiber.

21. The device of claim 19, wherein the pressure controlling part inhalations or discharges the operation fluid at a pressure of 1 to 15 psi.

22. The device of claim 19, wherein the pressure controlling part vibrates the balloon at a frequency of 1 to 100 Hz while maintaining a constant pressure.

23. The device of claim 19, wherein the pressure controlling part generates a vibration wave, and the vibration wave is delivered to the balloon through the operation fluid.

24. The device of claim 19, wherein at least one substance selected from the group consisting of an anti-inflammatory material, anti-infective material and anti-oxidation material having physiological compatibility is coated or impregnated on the surface of the balloon.

25. The device of claim 19, wherein the pressure controlling part controls the inhalation or discharge speed of the operation fluid so that the expansion and contraction speed of the balloon is 10 to 1000 μm/sec.

26. The device of claim 22, wherein the pressure controlling part vibrates the balloon, simultaneously when the laser system emits laser to the tissue through the optical fiber.

27. An electromagnetic energy application device for tubular tissue stricture, comprising:
a catheter;
a balloon catheter having an inner space interconnected with the catheter, connected to an end of the catheter enabling expansion and contraction;
a pressure controlling part inhalationing or discharging operation fluid to introduce the operation fluid into the balloon catheter or discharge the operation fluid from the balloon catheter through the catheter;
an optical fiber inserted into the balloon catheter penetrating through the catheter;
a laser system transmitting laser through the optical fiber; and
a location moving part withdrawing the balloon catheter.

28. The device of claim 27, wherein the optical fiber inserted into the balloon penetrating through the catheter is an optically diffusing fiber.

29. The device of claim 27, wherein the front end of the balloon catheter is formed in a sharp funnel shape, or the front and rear ends are symmetrically formed in a sharp funnel shape.

30. The device of claim 27, wherein the pressure controlling part inhalations or discharges the operation fluid at a pressure of 1 to 15 psi.

31. The device of claim 27, wherein the pressure controlling part vibrates the balloon catheter at a frequency of 1 to 100 Hz while maintaining a constant pressure.

32. The device of claim 31, wherein the pressure controlling part generates a vibration wave, and the vibration wave is delivered to the balloon catheter through the operation fluid.

33. The device of claim 31, wherein the pressure controlling part controls the inhalation or discharge speed of the operation fluid so that the expansion and contraction speed of the balloon catheter is 10 to 1000 μm/sec.

34. The device of claim 31, wherein the pressure controlling part vibrates the balloon catheter, simultaneously when the laser system emits laser to the tissue through the optical fiber.
